(19) United States
(12) Patent Application Publication
(10) Pub. No.: US 2025/0364108 A1
(43) Pub. Date: Nov. 27, 2025

(54) System and Methods Integrating Distributed Machine Learning Layers for Processing Multimodal Data Sets in Real Time to Optimize Outcomes

(71) Applicant: CorMetrix Inc., Portland, OR (US)

(72) Inventor: Saurabh Gupta, Portland, OR (US)

(21) Appl. No.: 19/215,220

(22) Filed: May 21, 2025

Related U.S. Application Data
(60) Provisional application No. 63/650,302, filed on May 21, 2024.

(51) Int. Cl. G16H 20/40; G16H 10/60

(52) U.S. Cl. CPC G16H 20/40; G16H 10/60

(57) ABSTRACT

An integrated medical platform with system and methods for enhancing medical procedures by rapid-data integration, artificial intelligence (“AI”) analyses with real-time, data-driven insights generated by machine learning models, which are executed in real time and continuously evolving to assist medical professionals in providing favorable outcomes. Other aspects of the integrated medical platform are configured to improve quality of procedures, automate regulatory headaches and streamline clinical coordination to improve outcomes and cost. In some embodiments, a unified tracking system is configured to track TAVR procedures introduces hospitals to an integrated, multimodal AI-enabled platform designed as an all-in-one platform configured to improve planning and care coordination of complex procedures. Smart data collection optimizes billing and streamlines registry data capture. It provides predictive clinical guidance powered by privacy-preserving federated deep learning and generative AI improves patient care.

### CROSS-REFERENCE TO RELATED APPLICATION

This application claims priority under USC § 119 (e) to U.S. Provisional Application No. 63/650,302, entitled “Integrated Medical Platform with Distributed Machine Learning Layers for Processing Multimodal Data Sets in Real Time to Optimize Outcomes” filed on May 21, 2024, the entirety of which application is herein incorporated by reference.

### BACKGROUND

### 1. Field of the Invention

The present invention relates to medical technologies and systems. More specifically, the present invention relates to systems and methods designed for enhancing medical procedures by integrating distributed machine learning layers for processing multimodal datasets (also referred to as data sets herein) in real time and generating artificial intelligence (“AI”) and machine learning models and executing them in real time, to continually evolve to deliver favorable outcomes.

### 2. Description of the Related Art

It is well known that current medical systems are antiquated and cumbersome. Each year, in the United States alone, doctors perform approximately 64 million medical procedures, ranging from tooth extraction to open heart surgery. Procedural medicine is a mammoth and growing market within healthcare, which is crippled by antiquated data management. Health systems drown in an ocean of data, yet lack the tools to derive insights, knowledge, judgment, and wisdom from the data, leading to subpar outcomes and needless increased spending.

Hospitals perform increasing volume of highly complex procedures, but lack tools to plan, facilitate, and optimize procedures. Health systems lack unified data tools and spend vast resources on care coordination. Current health systems do not adequately capture highest allowable reimbursement rates and struggle to comply with Medicare-mandated structured data reporting obligations for reimbursement. The structured training data database may comprise structured training data that has been annotated in accordance with a standardized format to facilitate efficient and consistent machine learning and AI model development based on the structured training data. For example, in some implementations, the structured training data may comprise collated annotated medical images, and medical terms/descriptions generated in accordance with the techniques described herein. To this end, the structured training data database may collate medical image data, medical report data and similar data that associates information that identifies and defines key data points in the medical image data to the medical report data using defined data structure, ontology, vocabulary for the respective types of data sets.

Clinicians lack data models for intelligent decision making. The medical device industry lacks real-time data to improve medical devices and procedures in response to clinical outcomes.

Consider one specific procedure, for example, a trans-catheter aortic valve replacement (TAVR). TAVR is a minimally invasive procedure that has surpassed open heart surgery in volume in the United States. This procedure alone is a high volume and growing complex procedure that requires multidisciplinary teams with large data sets. This procedure is burdensome and requires a data-intensive registry of structured data and reporting requirements. The data that must be provided for registry reporting is currently stored in a disorganized manner and requires significant human effort to collect for reporting.

Some systems that currently exist in the healthcare arena are Carta Healthcare, lumedx.com, and axisclinica.com, which systems are configured to simply extract structured data from patient charts. These systems do not address the critical need for clinical coordination of data inputs. Moreover, they fail to support clinical decision-making, lack advanced AI capabilities or multimodal data inputs (images, scans, and videos), do not contribute to optimizing billing and revenue capture, and, even more, fail to provide any insights to optimize outcomes.

Accordingly, there is a continuing need in healthcare for improved systems and methods that can intelligently build datasets, track and use them effectively to predict outcomes and modify user behavior and actions, whether the users are medical professionals, medical industry entities or patients. This background description provided herein is simply for the purpose of presenting the context of the disclosure.

### SUMMARY

The techniques introduced herein overcome the deficiencies and limitations of prior traditional systems and techniques, at least in part, by providing a system and methods configured to provide innovative artificial-intelligence-driven software algorithms, which are designed for use in conjunction with a user-friendly web application, to deliver comprehensive and enhanced medical outcomes that are quicker, safer, and consistent. The systems driven by artificial intelligence and machine learning models are configured to learn from comprehensive and disparate raw data and provide medical professionals (such as doctors) and hospitals with real insights and structured intelligence and form the core for next-generation practice of medicine and significantly improve decision-making for interventional strategies and related practices.

In accordance with some embodiments, the systems and methods integrate distributed machine learning layers for processing multimodal datasets (e.g., images, scans, and videos) in real time. The system and methods implemented provide an integrated platform, which serves as an all-in-one AI powered platform built to transform the efficacy and efficiency of medical procedures through rapid data integration and AI analyses that enables real-time data-driven insights delivered, for example, while performing medical procedures. In one implementation, data from every medical procedure performed trains the system to deliver better outcomes over time. The platform uses foundation models for the benefit of medical and patient communities to unlock data-driven diagnostic insights, improve quality of procedures, automate regulatory headaches, for example, legal and compliance, and streamline clinical coordination to improve outcomes and cost.

In accordance with some embodiments, generating AI insights in real time for performing medical procedures, for example, interventional procedures, improve patient outcomes.

In accordance with some embodiments, a unified tracking system for tracking TAVR procedures introduces hospitals to an integrated, multimodal AI-enabled platform designed as an all-in-one platform configured to improve planning and care coordination of complex procedures. Smart data collection optimizes billing and streamlines registry data capture. It provides predictive clinical guidance powered by privacy-preserving federated deep learning and generative AI improves patient care.

In accordance with some embodiments, the unified system provides an analytical framework configured to highlight patterns, flags, and anomalies, while identifying critical case metrics for review by medical professionals. This may involve quickly evaluating a medical case's merits to determine the strengths and weaknesses. Medical professionals may access integrated clinical notes, imaging, and laboratory information on a single distributed platform. In some embodiments, medical case materials may be distributed to different experts with one click.

In accordance with some embodiments, the unified system provides real-time data access, aggregation, and management with EHR integration. The system is implemented as a secure and user-friendly mobile platform, wherein the data remains in place. The platform has a standardized interface with access to mandated national registries and is a modular design that is easily scalable to other complex procedures.

In some embodiments, the unified system and methods described here provide predictive clinical guidance powered by privacy-preserving federated deep learning and generative AI foundation models. The system provides platform-agnostic collaboration between doctors and their teams with real-time coordination of multimodal data inputs. It facilitates appropriate billing and coding and automated regulatory reporting requirements with immediate return on investment for health systems and clinicians.

In accordance with some embodiments, the platform imports and collects data from a wide range of healthcare data sources including, but not limited to electronic health records, publicly available data, medical imaging data, social determinants of health and disease, video data, and derived data from the various available sources. The platform uses these data sources and powerful foundation models to deliver clinical insights derived from processing the data.

The features and advantages described herein are not all-inclusive and many additional features and advantages will be apparent in view of the figures and description. Moreover, it should be understood that the language used in the present disclosure has been principally selected for readability and instructional purposes, and not to limit the scope of the subject matter disclosed herein.

### BRIEF DESCRIPTION OF THE DRAWINGS

The disclosure is illustrated by way of example, and not by way of limitation, in the figures of the accompanying drawings in which, like reference numerals are used to refer to the same or similar elements.

FIG. 1 is a high-level block diagram, illustrating an example distributed environment in which the integrated and unified system (with prediction tools, software, or program) of the present implementation operates using distributed learning architecture and artificial intelligence algorithms and informatics in accordance with some embodiments of the present implementation of the invention illustrated here.

FIG. 2 is a block diagram illustrating example raw data structures constructed both in real-time and asynchronously and coupled to data collection portals and/or storage that are distributed over networks that are components in the integrated platform of FIG. 1 in accordance with some embodiments of the present implementation of the invention.

FIG. 3 is a block diagram illustrating the data sources and processed and structured data in accordance with some embodiments of the present implementation of the invention.

FIG. 4A is a block diagram of example hardware/software processing components of the integrated platform in accordance with some embodiments of the present implementation of the invention.

FIG. 4B is a block diagram of example hardware/software processing components of the integrated platform in accordance with some embodiments of the present implementation of the invention.

FIG. 5 is a block diagram illustrating the structural data sets coupled to the delivery optimization system of the integrated platform in accordance with some embodiments of the present implementation of the invention.

FIG. 6 is a block diagram illustrating the integrations of the integrated platform of the present invention with the application programming interface gateway.

FIG. 7 is a block diagram illustrating the application programming interface coupled to the integrated platform and methods (machine learning) in accordance with some embodiments of the present implementation of the invention.

FIG. 8A is a graphical representation of the data sources, deep learning network, and the predictive outcomes in accordance with some embodiments of the present implementation of the invention.

FIG. 8B is a block diagram illustrating example phases of the training process in the integrated platform.

FIG. 8C is a block diagram illustrating the various components within the cardiovascular interventional procedure module in accordance with some embodiments of the present implementation of the invention.

FIG. 8D is a block diagram illustrating the various components of the real-time device formation device in the integrated platform in accordance with some embodiments of the present implementation of the invention.

FIG. 9 is a high-level block diagram, illustrating the artificial intelligence subsystems for the reimbursement process in accordance with some embodiments of the present implementation of the invention.

FIG. 10 is a block diagram illustrating the raw medical imaging data flow in the integrated platform in accordance with some embodiments of the present implementation of the invention.

FIG. 11 is a block diagram illustrating the artificial intelligence subsystems in accordance with some embodiments of the present invention that direct the flow of raw internal and derivative data through neural networks to generate structured outputs that are used by predictive engines of the integrated platform.

FIG. 12 is a block diagram illustrating the artificial intelligence subsystems in accordance with some embodiments of the present invention that direct the flow of raw billing and coding data to generate billing/coded outputs that are directed by delivery engines to destination points.

FIG. 13 is a block diagram illustrating the artificial intelligence subsystems in accordance with some embodiments of the present invention that direct the flow of raw clinical data to generate structured clinical outputs that are coupled to clinical care predictive engines.

FIG. 14 is a block diagram illustrating the artificial intelligence subsystems in accordance with some embodiments of the present invention that direct raw video data flow through neural networks to generate video outputs for use by predictive engines of the integrated platform.

FIG. 15 is a block diagram illustrating the artificial intelligence subsystems in accordance with some embodiments of the present invention. These subsystems direct raw publicly available data flow through neural networks to generate outcome (e.g., video, audio, text, or composite data representations) outputs for use by the integrated platform's predictive engines. The data represented in outcome outputs may be classified as numerical data (including discrete and continuous), categorical (including nominal and ordinal), etc.

FIG. 16 is a block diagram illustrating the artificial intelligence subsystems in accordance with some embodiments of the present invention that direct raw electronic medical records data flow through neural networks to generate structured (or semi-structured) medical records (e.g., video, audio, text, or composite data representations) outputs for use by delivery engines of the integrated platform. The data represented in outcome outputs may be classified as numerical data (including discrete and continuous), categorical (including nominal and ordinal), etc.

FIG. 17 is a block diagram illustrating the artificial intelligence subsystems in accordance with some embodiments of the present invention that direct raw derivative data flow through neural networks to generate derivative outcome (e.g., video, audio, text, or composite data representations) outputs for use by predictive engines of the integrated platform. The data represented in outcome outputs may be classified as numerical data (including discrete and continuous), categorical (including nominal and ordinal), etc.

FIG. 18 is a block diagram illustrating the artificial intelligence subsystems in accordance with some embodiments of the present invention that direct raw regulatory compliance data flow through neural networks to generate regulatory/compliance structured outputs for use by delivery engines of the integrated platform.

FIG. 19 is a block diagram illustrating the artificial intelligence subsystems in accordance with some embodiments of the present invention that direct raw quality and structured reporting data flow through neural networks to generate reporting outputs for use by delivery engines of the integrated platform.

FIG. 20 is a block diagram illustrating the artificial intelligence subsystems in accordance with some embodiments of the present invention that direct raw social deterministic data (health and disease flow) through neural networks to generate social deterministic outputs for use by predictive engines of the integrated platform.

FIG. 21 is a block diagram illustrating the integrated layers of the global machine learning system, in the micro machine learning systems, the regional machine learning systems, and the global machine learning systems.

FIG. 22 is a flow chart illustrating the method of training the deep learning network models in accordance with the present implementation of the invention.

FIG. 23A is a flow chart illustrating the method in accordance with the present invention for identifying vendor devices for particular procedures (e.g., aortic valve placement) by determining a device appropriate for each patient based on precise measurements determined from patient-specific data. The method also maintains a dynamic index of vendor devices that is continuously updated once a device is approved by the Food and Drug Administration (FDA).

FIG. 23B is a block diagram illustrating a database or other storage structures (e.g., distributed over a network) for storing templates and data sets for the various medical procedures illustrated.

FIG. 23C is a flow chart illustrating the method in accordance with the present invention for identifying vendor devices for particular procedures by determining a device required for each patient based on precise measurements determined from patient-specific data. The method also maintains a dynamic index of vendor devices that is continuously updated once a device is approved by the Food and Drug Administration (FDA).

FIG. 24A is a flow chart illustrating the method for extracting multimodal patient-specific data, identifying subsets of data specific to a particular procedure, for example a TAVR procedure, segmenting relevant data sets and extracting and assembling them based on criteria identified in real time to create a composite image for either display to the medical team during procedure or for training neural networks.

FIG. 24B is a flow chart illustrating the method for extracting multimodal patient-specific data, identifying subsets of data specific to a particular procedure, for example any cardiovascular procedure, segmenting relevant data sets and extracting and assembling them based on criteria identified in real time to create a composite image for either display to the medical team during procedure or for training neural networks.

FIG. 25A is a flow chart illustrating the method for assembling data sets for compliance reporting for a TAVR procedure in real time and using them to train neural networks for automated assembly and formatting to expedite the process.

FIG. 25B is a flow chart illustrating the method for assembling data sets for compliance reporting for a specific cardiovascular procedure in real time and using them to train neural networks for automated assembly and formatting to expedite the process.

FIG. 26A is a flow chart illustrating the method for collecting, identifying, and automatically transforming data sets for a TAVR procedure into universal medical codes for billing purposes. The data sets are also used to train neural networks to automate the process.

FIG. 26B is a flow chart illustrating the method for collecting, identifying, and automatically transforming data sets for a specific cardiovascular procedure into universal medical codes for billing purposes and using the data sets to train neural networks to automate the process.

FIG. 27 is a flow chart illustrating the method for importing data from all sources in real time including, publicly available registry information, preprocessing data sets, anonymizing the data sets and pre-training models with publicly available scientific literature on risk factors for specific procedures and deploying models.

### DETAILED DESCRIPTION

While the present disclosure describes a system and methods for integrating distributed machine learning layers for processing multimodal datasets in real time and delivering outcomes in the context of example medical or healthcare environments, it should be understood that the platform and framework of tools described is capable of predicting outcomes in real time. In some implementations, the systems and methods are driven by artificial intelligence (“AI”) and configured as a cloud-based architecture that facilitates collection, storage, and distribution of complex clinical data across entire healthcare workflows, integrating imaging and relevant clinical and procedural data, to save time, eliminate redundancy, reduce expenses, accelerate pre-procedural insights, serve as a solution for procedure data management.

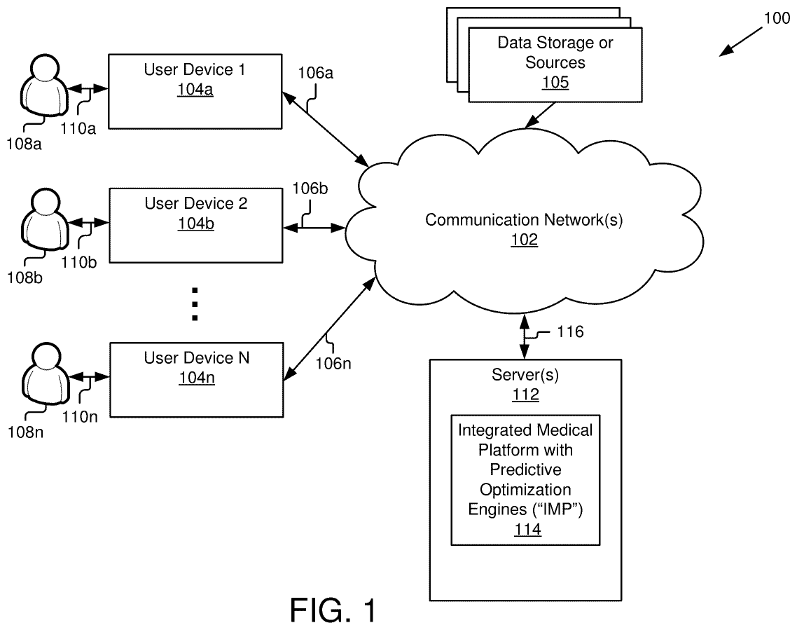

Referring now to FIG. 1, the integrated medical or healthcare platform(s) (otherwise referred to as a unified system or systems) with predictive optimization engines (hereinafter referred to as the “IMP” platform) and methods of the present invention are illustrated and described. The IMP platform is illustrated in an environment designated generally by reference numeral 100. The environment represents distributed environments (e.g., digital, server, cloud etc.) across any area, region, nation, or globally. In some implementations, the IMP platform may include one or more hardware servers, virtual servers, server arrays, storage devices and/or systems etc., and parts of the IMP platform may be centralized or distributed/cloud based. In some implementations, the IMP platform may include one or more virtual servers, which operate in a host server environment and access the physical hardware of the host server including, for example, a processor, a memory, applications, a database, storage, network interfaces, etc., via an abstraction layer (e.g., a virtual machine manager). The present implementation aims to advance the existing healthcare and medical systems, by incorporating artificial intelligence and machine learning technologies to unify medical data processes and improve outcomes.

The IMP platform environment 100 in accordance with the present implementation collects “raw” data from multiple sources (both public and patient specific), structures the raw data into structured data sets, and assembles multimodal data sets that are used to train neural networks to improve and predict outcomes. It should be recognized by those skilled in the art that “raw” data is any data originally generated by a system, device or operation, and has not been processed or changed in any way. Raw data may be obtained from a wide range of sources, such as machinery, monitors, instruments, sensors, surveys, log files, online transactions and countless other operations and places. Raw data is also referred to as source data, atomic data, and primary data. As will also be recognized by those skilled in the art, data scientists and analysts process the raw data to address specific questions and purpose and prepare it for presentation and/or further processing. The present implementation aims to enhance the efficiency of the global healthcare systems.

The present implementation is directed to a unified system and methods with artificial intelligence (AI-driven) software algorithms integrated with disparate web (and mobile) applications that are designed to provide access to entities involved in delivering healthcare and/or performing medical procedures. It should be recognized that the term “user” refers to any person or healthcare entity involved in delivering healthcare and performing the medical procedures.

In the following description, for purposes of explanation, numerous specific details are set forth in order to provide a thorough understanding of the technologies described. It will be apparent, however, that this technology can be practiced without some of these specific details. In other instances, structures and devices are shown in block diagram form in order to avoid obscuring the innovative aspects of the implementation. For example, the present technology is described in some implementations below with reference to particular hardware and software.

Various aspects of the present disclosure may be embodied as a method, a system, or a non-transitory, computer-readable storage medium having one or more computer-readable program codes stored thereon. Accordingly, various embodiments of the components of the present disclosure described may take the form of an entirely hardware embodiment, an entirely software embodiment comprising, for example, microcode, firmware, software, etc., or an embodiment combining software and hardware aspects that may be referred to herein as a “system,” a “module,” an “engine,” a “circuit,” or a “unit.”

Reference in this specification to “one implementation or embodiment” or “an implementation or embodiment” simply means that a particular feature, structure, or characteristic described in connection with the implementation or embodiment is included in at least one implementation or embodiment of the technology described. The appearances of the phrase “in one implementation or embodiment” in various places in the specification are not necessarily all referring to the same implementation or embodiment.

Some portions of the detailed descriptions that follow are presented in terms of algorithms and symbolic representations of operations on data bits within a computer memory. These algorithmic descriptions and representations are the means used by those knowledgeable in the data processing arts to most effectively convey the substance of their work to others in the art. An algorithm is conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “processing” or “computing” or “calculating” or “determining” or “displaying” or the like, refer to the action and processes of a computer system, or similar electronic computing device (such as or including the computer/processor), that manipulates and transforms data represented as physical (electronic) quantities within the computer system's registers and memories (such as or including the memory and data storage) into other data similarly represented as physical quantities within the computer system memories or registers or other such information storage, transmission or display devices.

It should be recognized that one of the greatest challenges is creating the infrastructure that can support healthcare. For the healthcare industry at large, there is currently no existing infrastructure for AI platforms. For example, large amounts of data is the core of AI-driven health care. Although data is the foundation upon which machine learning technologies are built, the increasing amounts of health care data are highly dispersed in siloes or proprietary formats that make it impossible to combine with external sources. Consider imaging analytics as an example of the complexity associated with using AI informatics tools to evaluate medical image data. For example, AI can be used in medical imaging to automatically identify and characterize features in images to make radiologists more efficient, minimize errors, and help medical professionals make their reports more quantitative, informative, and useful for patients. However, when trying to classify features in a computed tomography (CT) scan, the imaging algorithm may require anywhere from hundreds to millions of images to identify and learn consistent patterns and in all possible variants. Therefore, integration of AI based imaging analytics alone requires a unique infrastructure designed to facilitate storing, accessing, and processing such large data sets.

It should be recognized by those skilled in the art that not only are data access, storage, and processing demands are a critical issue, but interoperability between diverse systems involved in data storage, transfer, and processing is also a critical factor in enabling AI integration into healthcare systems. For example, infrastructure obstacles, such as an electronic health record (EHR) that is not operable with model development systems can make it difficult to access the specific data elements required to develop and train the AI models.

This type of fragmentation is a significant problem for hospitals or health systems when implementing clinical AI decision support tools that are not native to their current systems. In addition to data storage, access and interoperability requirements, to develop models configured to provide AI-based clinical solutions, it is imperative that the models must be trained and validated using massive amounts of accurately annotated training data. Machine learning algorithms are categorized into two broad classes, supervised and unsupervised. Unsupervised learning methods have been investigated and researched over the past few decades and, while encouraging, the maturity and robustness of these methods do not lend them as yet to the rigor required for routine clinical practice. Supervised learning techniques, however, are better suited due to recent computational and theoretical breakthroughs. In a supervised paradigm, the learning system is first given examples of data by which human teachers or annotators apply classification labels to collected data. The class labels are then used by the learning algorithm to adapt and change its internal, mathematical representation (such as the behavior of artificial neural networks) of the data and mapping to some predication of classification etc. The training consists of iterative methods using numerical, optimization techniques that reduce the error between the desired class labels and their algorithm's predictions. The newly trained models are then given new data as an input and, if trained adequately, can classify or otherwise provide assessment of novel data.

Because the supervised training paradigm is dependent upon varied data, it is imperative that training data is accurate and represents most of the variants the algorithm can perceive when new data is presented to it. For example, consider development of a diagnostic model configured to evaluate chest x-rays to classify them as normal versus abnormal. There may be hundreds of scenarios with different variables that designate an X-ray abnormal. Thus, to train a diagnostic model, a plethora of data is required that shows all the possible representations of all those different variables compared to representations that would be classified as normal. That would involve thousands or even millions of images, all of which must be labeled and annotated in a consistent manner.

Referring now to FIG. 1, the users designated by reference numerals 108a, 108b, through 108n, located at remote locations may communicate, as designated by signal lines 110a, 110b, through 110n, via their respective electronic devices. As illustrated, user device 1 is designated by reference numeral 104a, user device 2 is designated by reference numeral 104b, and user device N is designated by reference numeral 104n. Each or any of these electronic devices are in communication with one or more servers, designated as server(s) 112 including the IMP platform 114. It should be recognized that the IMP platform 114 is illustrated in a single block only for representation purposes. The IMP platform 114 may be distributed and implemented across many servers.

As illustrated in FIG. 1, the user devices 104a, 104b, and 104n are coupled via signal lines 106a, 106b, and 106n to the communication network (one or more) 102, which is coupled to the server 112 with the IMP platform 114 via signal lines, as designated by the signal line 116.

The communication network 102 represents any communication network or a combination of networks, for example, the internet, an intranet, a wired network, a wireless network, a communication network that implements Bluetooth® of Bluetooth Sig, Inc . . . a network that implements Wi-Fi® of Wi-Fi Alliance Corporation, an ultra-wideband communication network (UWB), a wireless universal serial bus (USB) communication network, a communication network that implements ZigBee® of ZigBee Alliance Corporation, a general packet radio service (GPRS) network, a mobile telecommunication network such as a global system for mobile (GSM) communications network, a code division multiple access (CDMA) network, a third generation (3G) mobile communication network, a fourth generation (4G) mobile communication network, a fifth generation (5G) mobile communication network, a long-term evolution (LTE) mobile communication network, a public telephone network, etc., a local area network, a wide area network, an internet connection network, an infrared communication network, etc., or a network formed from any combination of these networks.

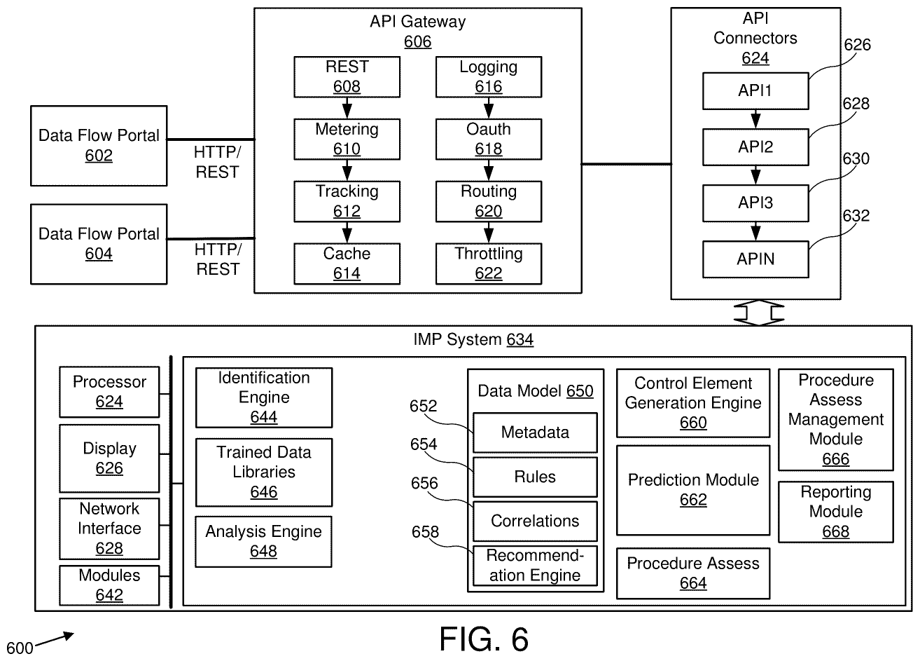

In some embodiments, the network 102 may comprise a local area network (LAN), a wide area network (WAN) (e.g., the Internet), and/or any other interconnected data path across which multiple devices may communicate. A network interface (e.g., network interface 628 in FIG. 6) may provide other conventional connections to the network 102 using standard network protocols such as TCP/IP, HTTP, HTTPS and SMTP as will be understood to those skilled in the art. In other embodiments, the network interface may include a transceiver for sending and receiving signals using WIFI, Bluetooth® or cellular communications for wireless communication.

FIG. 1 also illustrates a plurality of data storage or data sources designated by reference numeral 105. The data storage or sources 105 may be any data source, from which data is either obtained or stored in implementing the processes described here. Real world data refers to data relating to patient health status and/or the delivery of health care collected from myriad data sources. Example sources that provide real world data include, but are not limited to, electronic health records (EHRs), claims and billing activities, product and disease registries, data gathered from other sources, such as mobile phones, wearables, and smart watches, etc. The Food and Drug Administration (FDA) values real world data and evidence to support regulatory decisions.

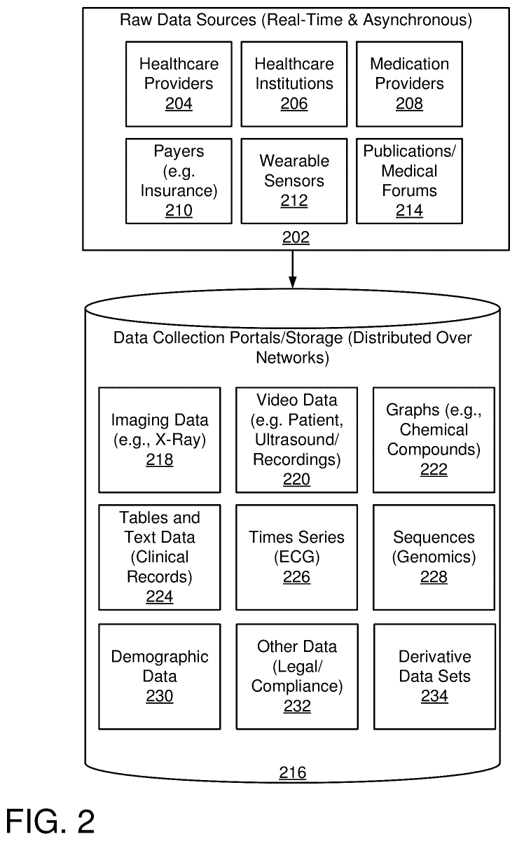

Referring now to FIG. 2, example raw data sources 202 are illustrated, which include healthcare providers 204, healthcare institutions 206, medication providers 208, payers (e.g., insurance) 210, wearable sensors 212, and publications/medical forums 214. Any of the raw data sources 202 may provide data flow either in real time or asynchronously to data collection portals 216 to storage distributed over the networks 102. The data collection portals may include, but are not limited to, imaging data 218, for example X-rays, video data 220, for example patients and ultrasound recordings, graphs 222, for example chemical compounds. Additional data collection portals 216 include tables and text data 224, including clinical records, times series (ECG) 226, sequences (genomics) 226, demographic data 230, other data 232, and derivative data sets 234. The derivative data sets 234 refer to data that reflects knowledge or information that is either inferred or derived from a data set, based on patterns mined by means of computations techniques, such as clustering, association rules, regression analyses, neural networks, reinforced learning, unsupervised algorithms, and more.

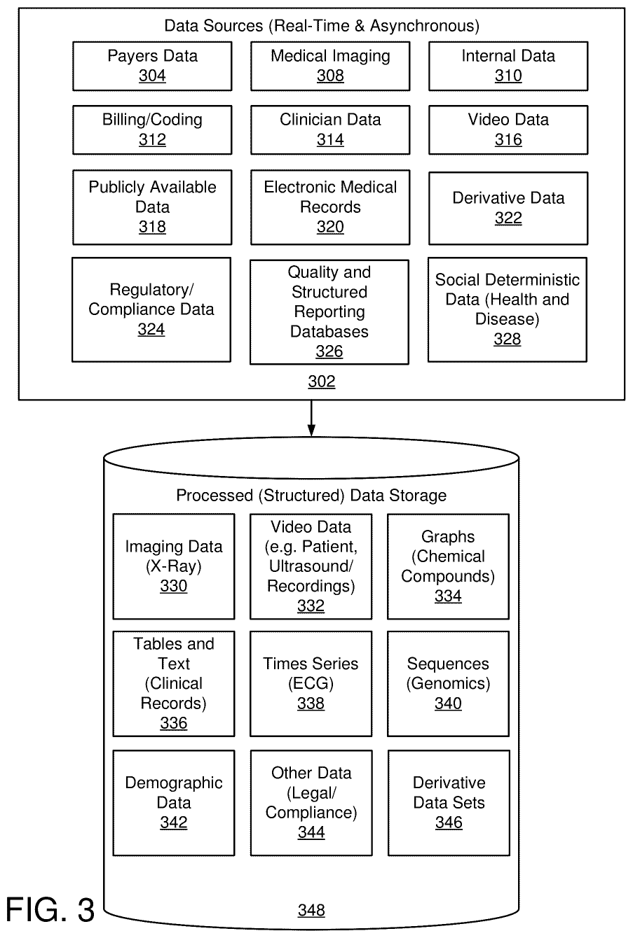

Referring now to FIG. 3, in some embodiments, example data sources 302, from which the IMP platform obtains data includes, but is not limited to, payers data 304, medical imaging data 308, internal data 310, billing/coding data 312, clinician data 314, video data 316, publicly available data 318, electronic medical records 320, derivative data 322, regulatory and/or compliance data 324, quality and structured reporting databases 326, and social deterministic data (health and disease) 328. The data obtained from any of the data sources 302 in real time or asynchronously, as illustrated, is processed into structured data and stored in data storage 348. The structured data sets are stored in data storage 348. In some embodiments, the processed data sets may include imaging data (X-Ray) 330, video data (e.g., patient, ultrasound recordings) 332, graphs (chemical compounds) 334, tables and text data (clinical records) 336. Times Series (ECG) 338, sequences (genomics) 340, demographic data 342, other data (legal or compliance) 344, and derivative data sets 346. In some embodiments, the structured data is configured in a standardized format for efficient access by software programs and humans. It may be configured in tabular form with rows and columns that clearly define data attributes. Computers may effectively process the structured data sets for insights due to its quantitative nature. In some embodiments, structured data lends itself to quick processing by the machine learning algorithms of the IMP platform. A structured query language (SQL) may be used to manage the structured data, to quickly input, search, and manipulate the structured data. In some instances, if unstructured data in its native formal is used, the unstructured data may be collected and preserved in data lakes. In such cases, the unstructured data remains undefined until required. Such unstructured data is adaptable, widens the data pool and enables data scientists to prepare and analyze only relevant data as it is used. The databases are constructed according to a federated architecture, to serve as a critical framework for managing complex distributed data systems. The global distributed architecture of the IMP platform enables seamless integration and necessary autonomy of the disparate data sources that are utilized.

In some implementations, the legal or compliance data 344 may comprise multiple entries, with each linked directly to its original source for quick verification and accuracy. The legal and compliance data may include FDA (Food and Drug Administration), HIPAA (Health Insurance and Portability and Accountability Act), GDPR (General Data Protection Regulation), ISO (International Organization for Standardization) and SOC2 (Service Organization Control 2) compliance processes. FDA requires compliance to regulations set to ensure the safety, efficacy, and quality of drugs and medical devices used by healthcare professionals. HIPPA is mandated for healthcare providers and SOC 2 is an optional framework for service organizations to demonstrate data security and privacy controls to their clients. In some implementations, the multimodal data sets are compiled by the modales and engines described here to define the scope of the compliance task, by identifying the healthcare entities covered under HIPAA, implement policies and procedures, conduct risk assessments, implement security measures, monitor and audit security controls, report breach events, and ensure vendors are in compliance with HIPAA requirements. In some implementations, for SOC 2 compliance, the multimodal data sets may be automatically generated by identifying applicable trust service criteria (TSCs), developing document policies and procedures related to the selected TSCs, implementing controls, conducting data audits, commission third-party audits and reports, monitor and maintain controls, and ensure that vendors are compliant with the SOC 2 requirements.

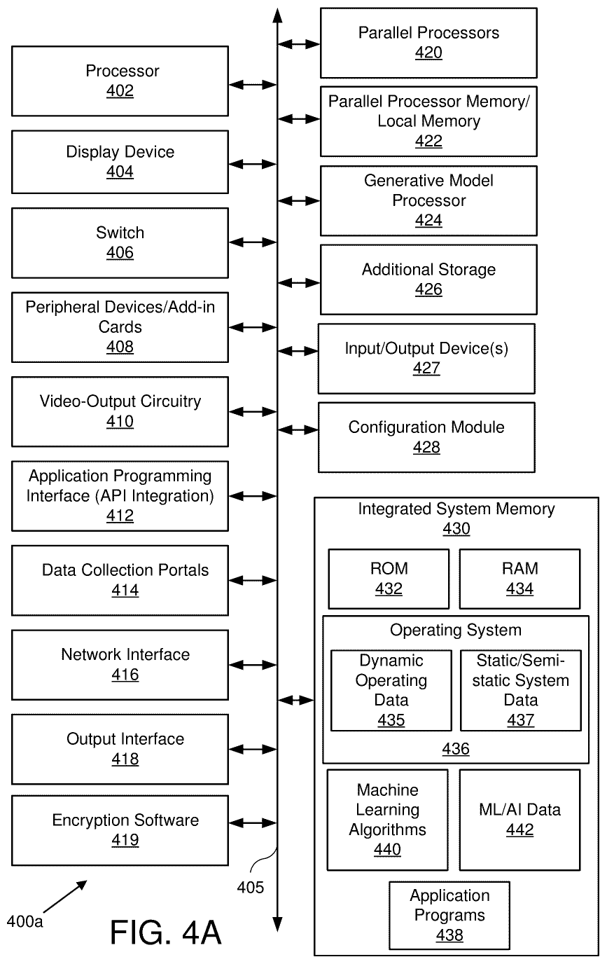

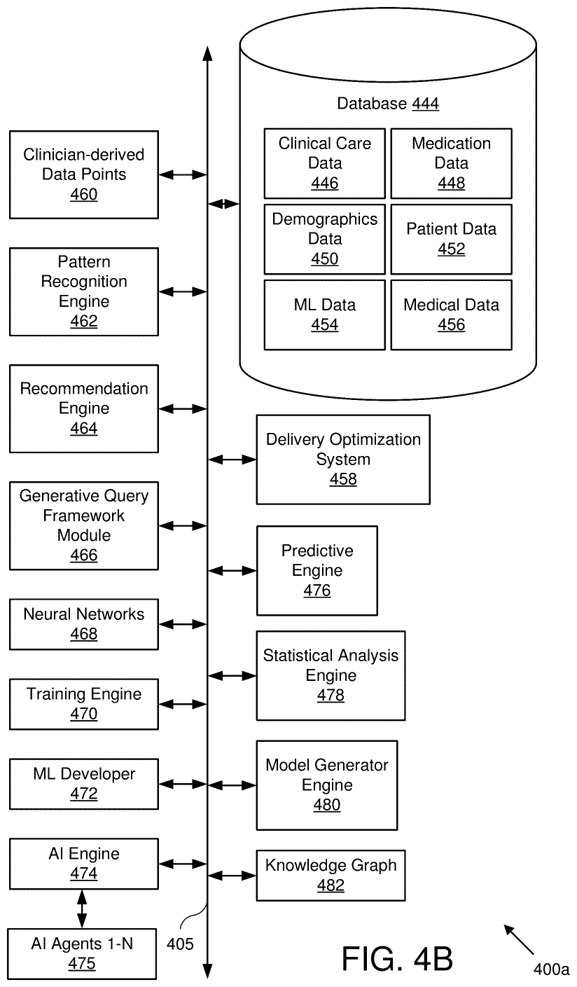

Referring now to FIG. 4A, the IMP platform with predictive optimization engines 114, has various hardware and software components, illustrated generally by reference numeral 400a. The hardware and software components 400a may include, but are not limited to, a processor 402 (including computing system for processing data and including any graphics processors by which graphical representations are generated for display), display device 404, a switch 406 (to switch between functions or programs), peripheral devices/add-in cards 408 to connect to peripheral or external devices and servers, video-output circuitry 410 coupled to the display device 404 via the bus 405, application programming interface (API integration) 412, data collection portals 414 (these are illustrated collectively here, but for each feature (data set) of interest or under consideration, there may be a separate portal dedicated to receive the data set of interest, network interface 416, output interface 418, and encryption software 419. The IMP platform 114 may comprise parallel processors 420 (several computing systems as required for machine learning systems), a parallel processor memory/local memory 422, a generative model processor 424, additional storage 426, an input/output (I/O) device(s) 427, a configuration module 428, and an integrated system memory 430. All the components that are illustrated in this figure are coupled via the bus 405. The integrated system memory 430 may comprise a ROM (Read-only) memory 432 and a RAM (Random-access) memory 434, an operating system 436, for dynamic operating data 435 and static/semi-static system data 437, machine learning (“ML”)/artificial intelligence (“AI”) data 442, and application programs 438. It should be recognized that the ML/AI data 442 is illustrated by a single block simply for ease of illustration, but this may be implemented on various databases in a distributed architecture.

The processor 402 is configured to execute the computer program instructions defined by the various components illustrated. The processor 402 refers to any one or more microprocessors, central processing unit (CPU) devices, finite state machines, computers, microcontrollers, digital signal processors, logic, a logic device, a user circuit, an application specific integrated circuit (ASIC), a field-programmable gate array (FPGA), a chip, etc., or any combination thereof, capable of executing computer programs or a series of commands, instructions, or state transitions. In some embodiments, the processor 402 is implemented as a processor set comprising, for example, a programmed microprocessor and a math or graphics co-processor. The system here is not limited to employing the processor 402. In some embodiments, the processor 402 may employ controllers or microcontrollers. The processor 402 executes the instructions, for example, any of the engines and software modules described here.

It should be recognized that the data collection portals 414 as represented, include any number of portals for receiving data gathered from many sources as described above with respect to the other figures. All data required for processing at the IMP platform 114 is gathers continuously vial the data collection portals 414.

The network interface 416 may be a network interface controller, a network interface card, a network adapter or any such hardware component that is designed to allow computers to access an interconnection network for communication and synchronization purposes. The network interface 416 is configured to provide two different kinds of interfaces, one toward the computer (host) side and one toward the network side. The network interface 416 translates the protocol of the host interface to the network protocol and vice versa, and translates between the different physical media. In some embodiments, in the communication network 102, the network interface 416 represents the end points of the network 102. At these endpoints, the network packets are injected into, respectively, or retrieved from the network 102. As all end points of the network 102 are uniquely addressable, each network interface 416 is assigned a unique number. Modems, cable modem, and ethernet cards are just a few of the currently available types of network adapters. For example, the network adapter may be a network interface module coupled to the network 102 by a signal line and the bus 405 as illustrated. The network interface 416 may include ports for wired connectivity such as but not limited to USB, SD, or CAT-5, etc. The network interface may link to the processor 402 and to the network 102 that may in turn be coupled to other processing systems.

The processor 402 as illustrated is a computer or data processing system suitable for storing and/or executing program or executable code in any of the modules or engines described here. The processor 402 includes at least one processor coupled directly or indirectly to memory elements (integrated system memory 430 or such data storage) through the system bus 405. The memory elements include the integrated system memory 430 utilized during actual execution of the program code, bulk storage, and cache memories (e.g., see cache), which provide temporary storage of at least some program code in order to reduce the number of times, code must be retrieved from bulk storage (data storage) during execution. Input/output or I/O devices (including, but not limited to keyboards, displays, pointing devices, etc.) are coupled to the system either directly or through intervening I/O controllers. A display device representative of all these elements is illustrated and designated by reference numeral 404. A display as illustrated may include a mouse, keyboard, video card (video monitor), sound card (with speakers), network card, and a printer. The display device 404 as illustrated represents distributed functionalities. The display device 404 serves to display any information, data, or control functions of the hardware and software components 400a. For example, the display device 404 may be used to display selective data populated by predictive models generated by the generative model processor 424. In some embodiments, users may have an application on their telephones or desktop web browser, by which they may display outcomes or predictive recommendations provided by the various hardware and software components. The outcomes or predictive recommendations build on the patterns identified in the various data sets, identified in the prior figures.

In some embodiments, the IMP platform 114 of the present implementation may use a decision-based model to predict the likelihood of medical outcomes, flag any health risks or need for intervention before performing procedures. The IMP platform 114 has a metadata-based framework that involves a series of decisioning queries and executes functions based on answers to the decisioning queries. The framework creates critical points that may be designated as “good,” “neutral,” or “concerning,” and recommends various levels of interventions based on combinations of features. This metadata approach also provides system flexibility to add more functions as more data is collected.

In some implementations, the encryption software 419 is software designed to meet the Health Insurance Portability and Accountability Act (HIPAA) requirements for protecting patient information (PHI). The encryption software 419 includes strong encryption algorithms designed to safeguard data during transit on communication lines and at rest. The encryption software algorithms integrate easily with the Electronic Health Record (HER) systems and other healthcare tools. The encryption software 419 permits sharing of data with authorized parties and uses role-based access controls, secure messaging, and end-to-end encryption to protect patient information. In some implementations, anonymization software with anonymization protocols may be used to select and anonymize subsets of sensitive data, which may be stored with an identifier for a particular anonymization protocol. Control software executed by the processor may be configured to receive the anonymized subset of data and the identifier to analyze and execute the functions designated for the subsets of data with the framework described here.

In some embodiments, the generative model processor 424 uses neural networks 468 as further described below to process the vast amounts of data collected in real time. The neural networks 468 rely on training data that is continuously (or asynchronously) updated to learn and improve their accuracy over time, classifying and clustering data at high velocity. Each of the neural networks 468 consists of layers of nodes or artificial neurons, in an input layer, one or more hidden layers, and an output layer. Each node connects to others and has an associated weight and threshold. If the output of any individual node is above the specified threshold value, that node is activated, sending data to the next layer of the neural network 468. As will be recognized, neural networks 468 are also referred to as deep learning algorithms, depending upon the depth of the layers. The generative model processor 424 develops frameworks for interpreting metadata that is entirely metadata-driven to anticipate rapid evolution. The application programming interface 412 integrates the backend API with the frontend that may pass decisions between them.

Referring now the FIG. 4B, the IMP platform 114 (FIG. 1) designated by reference numeral 400a in this figure is configured to accomplish data processing and analysis (by the computing systems illustrated). The IMP platform processes a vast array of clinician-derived data points 460 (including any anonymized patient or other data), initially labeled by clinical experts, to unveil complex, multifactorial medical correlations. FIG. 4B illustrates the clinician-derived data points 460, the pattern recognition engine 462, the recommendation engine 464, the generative query framework module 466 (for framing queries), the neural networks 468, the training engine 470 (e.g., designed to continuously train models), the Machine Learning (ML) developer 472, the AI (Artificial Intelligence) engine 474, the Database 444, the delivery optimization system 458, the predictive engine 476, the statistical analysis engine 478, the model generator engine 480, and the knowledge graph 482. Knowledge graph 482 represents a network of entities, such as objects, events, situations, or concepts and illustrates the relationship between them. Knowledge graph 482 consists of nodes, edges, and labels, wherein any object, place, or person is represented by a node, for example, a hospital may be represented by a node. The edge defines the relationship between the nodes, for example, the relationship between a person who is a patient customer of the hospital and so on.

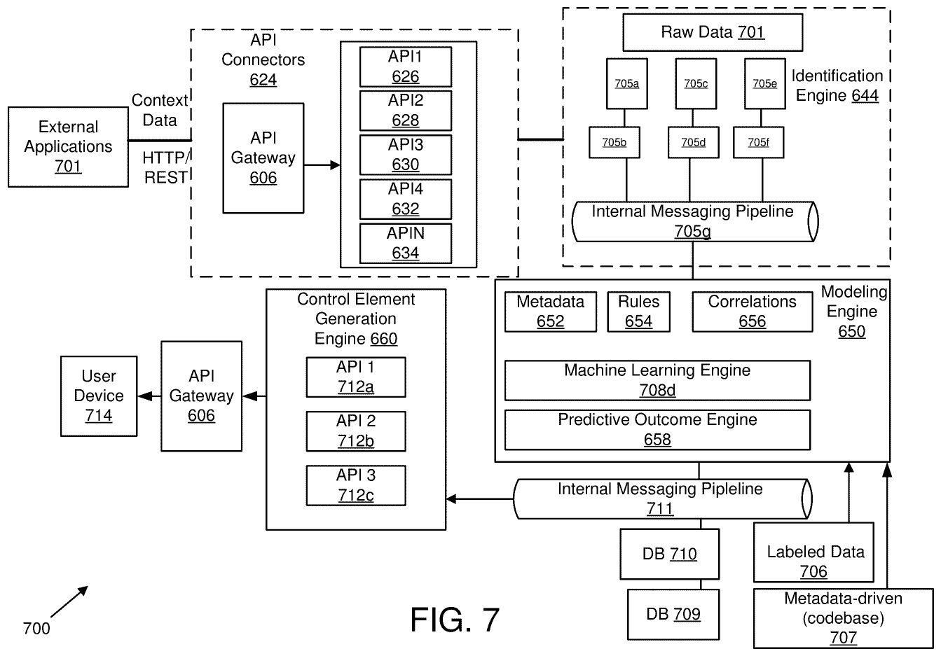

In some embodiments, the database 444 may include data such as clinical care data 446, medication data 448, demographics data 450, patient data 452, ML data 454, and medical data 456, and the like. It should be recognized that the database 444 may comprise a plurality of databases, distributed over regions, that are operationally linked. It should also be recognized by those skilled in the art that any number of databases may be used to store the data collected in real time (e.g., DB 709 and 710 shown in FIG. 7). The datasets retrieved or compiled may be labeled, classified and stored in the different databases.

The IMP platform model development processes involve data visualization, dimension reduction techniques, and advanced algorithms for feature selection. The IMP platform's predictive modeling functions may combine traditional statistical analyses with supervised neural network technology, enhancing predictive accuracy and allowing continuous model adaptation and evolution. Specifically, the IMP platform performs extensive data analyzes to identify medical-influencing patterns, leading to product or tool creation. These functions involve parallel paths of data exploration and software engineering, with a focus on statistical analyses to determine key medical contributors, model refinement using decision-based predictions, and neural network development for categorizing medical likelihoods. It should be recognized that supervised machine learning models are trained with labeled data sets, which allow the models to learn and become more accurate over time. An entirely metadata-driven codebase (707 in FIG. 7) and application programming interface (API) are used to develop assessments to ensure adaptability and integration with user interfaces that are intuitive. Key stages in the model's development include data visualization for procedure-based analysis and dimension reduction techniques like principal component analysis, K-means clustering, and advanced algorithms for critical data selection.

The predictive engine 476 performs the predictive modeling. The IMP platform's predictive capabilities (in the predictive engine 476) are anchored in its advanced analytical framework. The models generated utilize a high-performing and viable approach that develops models with limited yet complex processed datasets. The models employ an augmented approach, combining traditional statistical analysis (by the statistical analysis engine 478) with a supervised neural network (by neural network 468), to yield more precise results. The IMP platform's predictive accuracy stems from its ability to process and interpret intricate patterns (by pattern recognition engine 462 in FIG. 4B) within the data, a function enhanced by its entirely metadata-driven codebase (707 in FIG. 7). This flexible framework allows for continuous adaptation and evolution of the models, accommodating unsupervised analysis as additional data (stored in additional storage 426 in FIG. 4A or databases “DB” in FIG. 7, designated by numerals 709 and 710) becomes available.

Referring now to FIG. 7, the databases (DB) 710 and 709 couple to an internal messaging pipeline 711, which couples to a modeling engine 650 and a control element generation engine 660. It should be recognized that the IMP platform 114 (in FIG. 1) maintains data privacy and security measures. Data privacy is maintained in the IMP platform's approach and functions, encompassing ethical norms, security protocols, operational methods, and strategic technical decisions. The IMP platform uses cloud services (e.g., Google cloud services in the network 102), selected for their compliance with HIPAA standards, for secure handling of medical-grade data. The IMP platform's data management includes rigorous access control, limiting full data access to only authorized individuals, and enforcing a mandatory identification process before any data analysis.

Furthermore, the IMP platform adheres to a stringent anonymization protocol for sensitive data (e.g., by the clinician-derived data points 460 in FIG. 4B) used in machine learning model development (by ML Developer 472), ensuring removal of all personal identifiers relating to any patients. The platform operations align with the highest standards of data privacy and security, enhancing the integrity and accuracy of the machine learning models. It should be recognized that the IMP platform proactively integrates user data protection and privacy across all jurisdictions.

The user interface and interaction functions performed include web-based IMP models with user-friendly digital tools, providing customized reports and tailored procedure protocols. In some embodiments, the application is configured to provide users 108a, 108b, through 108n (in FIG. 1) with a customized summary report across critical categories relating to each medical procedure. This report not only indicates factors that are satisfactory or need attention but also offers tailored preventive care advice and flags potential medical issues. Moreover, the application may identify each patient's likelihood of developing a potential post-procedure condition.

The digital decision tools of the present IMP platform 114 are designed with utmost consideration for user privacy. In some embodiments, access is available via a website. Users may initiate their personalized functions by clicking “START NOW.” It should be recognized that insights are available for patients, clinicians, providers, clinics, device manufacturers and payors. The IMP platform empowers health professionals with early access to critical medical information, thereby ensuring successful outcomes. The generative model insights enable medical practitioners to ensure successful outcomes. The drawing figures illustrate the system architecture, factors analyzed, processing methodology etc.

Referring again to FIG. 4A, the IMP platform 114 comprises the network interface 416 configured to couple to each of data resources (e.g., illustrated in FIGS. 2 and 3), from which data may be compiled in real time, either continuously or at designated times or intervals (i.e., asynchronously). Different types of datasets (generated or identified via data collection portals 414) are compiled by dynamic data compilers in these data collection portals 414. Referring now to FIG. 4B, the IMP platform 114 (FIG. 1) is configured to search for patterns (by pattern recognition engine 462) using artificial intelligence (“AI”) engine 474 or consider pre-determined rules and triggers set within the IMP platform when considering data from sources. The IMP platform determines and uses the patterns, rules, and triggers to offer recommended actions (by the recommended engine 464) such as providing specific feedback, targeted protocols, etc. In some implementations, the AI engine 474 (e.g., with generative query framework module 466) is configured to control conversational agents between distributed systems designed to share data instantly, without delving through stored PDF documents with keywords. For example, a query from a particular medical professional may inquire “when did the patient first complain of pain? Yet another query may request “were there complications after surgery?” Yet another query may request “what medications was the patient taking?” Any of these critical questions may surface at any stage of a medical examination or procedure. In some implementations, the AI engine 474 is configured to interact with and control multiple different AI agents 1-N, indicated by reference numeral 475, which may control tasks, make decisions, and adapt based on data and user (e.g., patient or doctor) feedback. In some implementations, the AI agents 475 are designed to retain data from previous interactions and maintain their state across different tasks and situations. The AI agents 475 facilitate quick processing by analyzing the perceived environment based on interactions and planning next actions by using actuators or such control mechanisms. The AI agents 475 may be structured in a hierarchical order and follow condition-action rules based on their understanding of context.

Referring to FIG. 4A, the integrated system memory 430 (in FIG. 4A) is a typical general purpose computer system that includes computer-readable and writeable nonvolatile recording medium, of which, a magnetic disk, a flash memory and tape are examples. A ROM 432 and RAM 434 are illustrated as examples. The integrated system memory 430 operably holds the operating system 436 and application programs 438. The integrated system memory 430 also holds machine learning algorithms 440 (e.g., which may be modified or updated by human input) and ML/AL data 442. The ML/AI data 442 may include structured, unstructured, and device-level data across environments. It should also be understood that the implementation is not limited to the particular input devices, output devices, or memory systems used in combination with the computer system or to those described herein. Nor should the implementation be limited to any particular computer platform, processor, or high-level programming language.

The processor 402 processes data signals and program instructions received from the integrated system memory 430. For example, the processor 402 may comprise an arithmetic logic unit, a microprocessor, a general-purpose controller or some other processor array programmed in accordance with the present implementation to perform computations and provide electronic display signals to the display device 404. The processor 402 is coupled to the bus 405 for communication with the other components. The processor 402 processes data signals and may comprise various computing architectures including a complex instruction set computer (“CISC”) architecture, a reduced instruction set computer (“RISC”) architecture, or an architecture implementing a combination of instruction sets. Although only a single processor 402, multiple processors may be included in a single location or in distributed locations. It should be recognized to one skilled in the art that other processors, operating systems, sensors, displays and physical configurations are possible.

The integrated system memory 430 is non-transitory storage medium. The memory 430 stores the instructions and/or data which may be executed by the computer/processor 402. In some embodiments, the instructions and/or data stored on the system memory 430 comprises code for performing any and/or all of the techniques and functionalities described herein. The integrated system memory 430 may be a dynamic random-access memory (DRAM) device, a static random-access memory (SRAM) device, flash memory or some other memory device known in the art. The integrated system memory 430 may store a web browser with JavaScript or other programming language for conducting any functions that require access to remote networks or libraries. In some embodiments, the integrated system memory 430 may include a cache memory 614 (FIG. 6) coupled to the processor 402. The cache memory 614 (FIG. 6) may be a random-access memory (RAM) that the processor 402 may access more quickly than it can access regular RAM. This cache memory 614 in the integrated system memory 430 is typically integrated directly with the processor chip or may be on a separate chip that has a separate bus interconnect with the processor 402. The cache memory 614 is used to reduce the average time to access data from the main memory 430 or data storage. The cache 614 is a smaller and faster memory, which stores the program instructions that are frequently referenced by the software during operation. Fast access to these instructions increases the overall speed of the software program. In operation, as the processor 402 processes data, it looks first in the cache memory 614 for instructions and if it finds the instructions there (e.g., from a previous reading of the data), it does not conduct a more time-consuming reading of data from the larger memory in the system memory 430 or other data storage devices. In some instances, multi-tier or multilevel caching, with different levels providing greater efficiency may also be used.

In some embodiments, a non-transitory, computer-readable storage medium, for example, the integrated system memory 430 is deployed in the IMP platform 114 (FIG. 1), for storing computer program instructions defined by the engines and modules. As used herein. “non-transitory computer-readable storage medium” refers to all computer-readable media, for example, non-volatile media, volatile media, and transmission media, except for a transitory, propagating signal. Non-volatile media comprise, for example, solid state drives, optical discs or magnetic disks, and other persistent memory volatile media including a dynamic random-access memory (DRAM), which typically constitute a main memory. Volatile media comprise, for example, a register memory, a processor cache, a random-access memory (RAM), etc. Transmission media comprise, for example, coaxial cables, copper wire, fiber optic cables, modems, etc., including wires that constitute a system bus coupled to the processor 402. The processor 402 is operably and communicatively coupled to the integrated system memory 430 for executing the computer program instructions defined by the modules, for example, any of the engines and modules described here. The integrated system memory 430 is used for storing program instructions, applications, and data. The integrated system memory 430 is, for example, a random-access memory (RAM) or another type of dynamic storage device that stores information and instructions for execution by the processor 402. The integrated system memory 430 also stores temporary variables and other intermediate information used during execution of the instructions by the processor 402. The IMP platform 114 further comprises read only memories (ROMs) or other types of static storage devices that store static information and instructions for execution by the processor 402. In some embodiments, the various modules, of the system are stored in the integrated system memory 430.

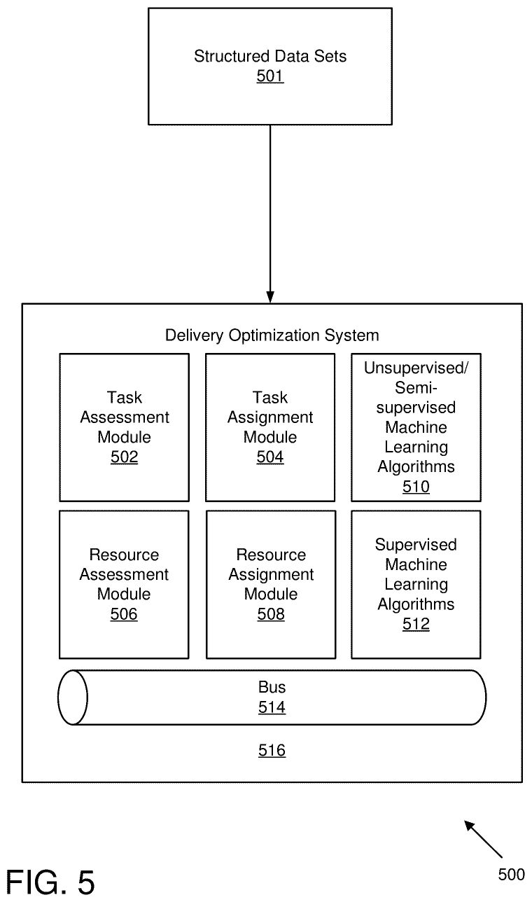

Referring now to FIG. 5, as designated generally by reference numeral 500, the architecture of the delivery optimization system 516 in communication with structured data sets is illustrated. The delivery optimization system 516 includes a task management module 502, a task assignment module 504, unsupervised/semi-supervised machine learning algorithms 510, a resource assessment module 506, a resource assignment module 508, and supervised machine learning algorithms 512 are illustrated. Data between each of these modules flow via a bus 514.

As illustrated by way of example in FIG. 6 (as designated generally by reference numeral 600), the IMP platform 114 in some embodiments, further comprises the data bus, the processor 624, the display 626, the network interface 628, and modules 642. The data bus permits communications between the modules, for example, any of the engines and modules described here. The display 626, via a graphical user interface (GUI), as generated by the dynamic user interface generator is configured to display information, display interfaces, user interface elements such as checkboxes, input text fields (in surveys, summaries), etc., for example, for allowing a user to configure message templates and personalization preferences for facilitating the dynamic generation of medical content (e.g., risks, alerts, summaries, predictions, etc.) and other targeted, feature control elements for target users. The system renders the GUI on the display 626 for receiving inputs from target users. The GUI comprises, for example, online web interfaces, web-based downloadable application interfaces, mobile-based downloadable application interfaces, etc. The display 626 displays the GUI.

The network interface 628 enables connection of the IMP platform components to the network 102 (in FIG. 1) via the API system 700 (in FIG. 7). In some embodiments, the network interface 628 is provided as an interface card also referred to as a line card. The network interface 628 may be, for example, one or more of infrared interfaces, interfaces implementing Wi-Fi® of Wi-Fi Alliance Corporation, universal serial bus interfaces, FireWire® interfaces of Apple Inc., Ethernet interfaces, frame relay interfaces, cable interfaces, digital subscriber line interfaces, token ring interfaces, peripheral controller interconnect interfaces, local area network interfaces, wide area network interfaces, interfaces using serial protocols, interfaces using parallel protocols, Ethernet communication interfaces, asynchronous transfer mode interfaces, high speed serial interfaces, fiber distributed data interfaces, interfaces based on transmission control protocol/internet protocol, interfaces based on wireless communications technology such as satellite technology, radio frequency technology, near field communication, etc. The modules 642 of the IMP platform 114 (in FIG. 1) comprise, for example, input/output (I/O) controllers, input devices, output devices, fixed media drives such as hard drives, removable media drives for receiving removable media, etc. Computer applications and programs are used for operating the subsystems 600 illustrated here of the IMP platform 114. The programs are loaded onto fixed media drives and into the integrated system memory 430 (in FIG. 4A) via the removable media drives. In some embodiments, the computer applications and programs are loaded into the integrated system memory 430 directly via the network 102.

The network interface or adapter 628 facilitates communications between the software servers via the network 102 (FIG. 1) for remote processing or linking the distributed remote user devices or systems system controller (in the processor). The network interface 628 or adapter may link to other remote computers with memory storage. The system controller is coupled to the processor 624 and is configured to gather data from target users. The data may be provided or received in real time in various ways. The system controller executes the applications configured to operate the functions of the system and runs the operating system (“OS”) configured to manage all of the software and hardware on the computer. As is recognized by those skilled in the art, the operating system 436 performs all the system computing tasks, such as controlling the backing store and peripherals such as scanners and printers, handling the transfer of programs in and out of memory, organizing the use of the memory between programs, organizing the processing time between programs and the users, and maintaining security and access rights of users 108a, 108b, through 108n. The applications also referred to as an application program or application software, perform the specific functions directly for the end users, or in some cases, for another application.

Referring now to FIGS. 6 and 7, an architectural diagram 600 illustrates an exemplary implementation of the modules of the IMP system 634 (designated as 700 in FIG. 7) for executing outcomes and predictions for a target user via an application programming interface. In some embodiments, the various modules of the IMP subsystem 700 disclosed herein are deployed in the IMP platform 114. The IMP subsystem 700 accesses multiple external applications 701 (FIG. 7) associated with data providers and service providers respectively, via the application programming interface (APD) system 700. The API system 700 connects to the external applications 701 (providing data sets) via the network 102, for example, a short-range network or a long-range network. The API gateway 606 of the API system 700 acts as a single point of entry for retrieving multiple data sets from the external applications 701. In some embodiments, the API gateway 606 uses a REST web services architecture to access the external applications 701. Context data from features or factors may be taken from the external applications 701 and conveyed via API connectors 612. Furthermore, the API gateway 606 handles operations, for example, metering 610, tracking 612, logging 616 (e.g., comprehensive logging for all activities), authentication 618, routing 620, and throttling 622 to execute communications between the external applications 701 and the API connectors 624. The API gateway 606 stores operational data in a cache 614. The API gateway 606 connects to the API connectors 624 of the API system 600 comprising multiple APIs, for example, “API1” 626, “API1” 628, “API1” 630. “APIN” 632, etc., that provide access to different external applications 701 (to gather data sets) via a load balancer. It should be recognized by those skilled in the art that the load balancer distributes and balances the workload in the IMP system 700 via the API gateway 608.

The IMP subsystem 600 (in FIG. 6) is a computer system programmable using high-level computer programming languages. In some embodiments, the IMP subsystem 600 is implemented using programmed and purposeful hardware. In some embodiments, the subsystem 600 is accessible to users, for example, through a broad spectrum of technologies and user devices such as smartphones, tablet computing devices, endpoint devices, etc., with access to the network 102. In some embodiments, the IMP subsystem 600 is implemented in a cloud computing environment. In some embodiments, the IMP subsystem 600 is a cloud computing-based platform implemented as a service for providing medical assessments and predictions to a target user. In some embodiments, the IMP subsystem 600 is implemented as an on-premise platform comprising on-premise software installed and operated on computers on the premises of an organization.

In some embodiments, the IMP subsystem 600 includes a machine learning engine 708d (FIG. 7), and therefore, more than one specifically programmed computing system is used for assessing medical-related behavior of a target user. Users, for example, patients with specific conditions, may be expected to behave a certain way to meet the medical classifications for particular diagnosis.

Referring to FIG. 6, in some embodiments, the IMP subsystems disclosed herein further comprise an identification engine 644, trained data libraries 646, an analysis engine 648, data model 650, which comprises metadata 652, rules 654, correlations 656, and a recommendation engine 658. The IMP system 634 further comprises a control element generation engine 660, a prediction module 662, a procedure assessment (“assess”) module 664, a procedure assessment (“assess”) management module 666, and a reporting module 668. Referring now to FIG. 7, the control element generation engine 660 further comprises API 1 (712a), API 2 (712b), and API 3 (712c).

Referring to FIG. 6 again, the reporting module 668 is in communication with the procedure assessment (“assess”) module 664 and the prediction module 662, for generating reports for the target users. For example, in some embodiments, the reporting module 668 may generate and render multiple reports for medical professionals with medical-related associations for particular patients and relevant medical expectations. The reporting module 668 may also generate online reports with charts and graphs to provide complete visibility of the entire medical procedure process implemented by the IMP system 634 disclosed herein. In some embodiments, the reporting module 668 may be configured to perform an inference analysis, for example, by determining the percentage of users who are not meeting the medical threshold levels on progress for particular procedures performed.

In some embodiments, the modules may further comprise medical (“A”, “B.” etc.) labeled data 706 for automatically generating medical classification data based on the patient behavioral models dynamically generated for tracking patient progress before or after procedures are performed.

It should be recognized by those skilled in the art that a module, or an engine, or a unit, as used herein, refers to any combination of hardware, software, and/or firmware. As an example, a module, or an engine, or a unit may include hardware, such as a microcontroller, associated with a non-transitory, computer-readable storage medium to store computer program codes adapted to be executed by the microcontroller. Therefore, references to a module, or an engine, or a unit, in an embodiment, refers to the hardware, which is specifically configured to recognize and/or execute the computer program codes to be held on a non-transitory, computer-readable storage medium. Furthermore, in another embodiment, use of a module, or an engine, or a unit refers to the non-transitory, computer-readable storage medium including the computer program codes, which is specifically adapted to be executed by the microcontroller to perform predetermined operations. In another embodiment, the term “module” or “engine” or “unit” refers to the combination of the microcontroller and the non-transitory, computer-readable storage medium. Often module or engine boundaries that are illustrated as separate commonly vary and potentially overlap. For example, a module or an engine or a unit may share hardware, software, firmware, or a combination thereof, while potentially retaining some independent hardware, software, or firmware. In various embodiments, a module or an engine or a unit includes any suitable logic.

Referring to FIG. 6, in some embodiments, the trained data libraries 646 may comprise, for example, a library of medical scenarios on various procedures, a compliance mapping library, a group intelligence library, etc. In some embodiments, the trained data libraries 646 store micro-messages containing a few lines of text, short interactive content, imaging data, interactive videos, etc., that are used by the control element generation engine 660. In some embodiments, the data model 650 utilizes data from the trained libraries 646. The control element generation engine 660 renders targeted, trained-data-based control elements to one or more of the user applications on a user device 714 of a user 108 via API integrations. For example, when the user 108 attempts to take an action via an email client, the control element generation engine 660 may render a real-time cue “Are you sure you want to handle this task in this way?” within the email client. In another example, the control element generation engine 660 may send an email with a notification message “Here is some additional information for you to consider” and a link to access medical procedure content, medical assessments on patients, or medical prediction or outcome data and provide this to the user 108 (e.g., a medical professional).

In yet another example, the control element generation engine 660 may send a notification message “Did you address the medical procedure yet? Here is a recommendation” through a collaboration app such as the Slack® app. In another example, the control element generation engine 660 may send a notification message “Do you want to download all outcome details?

The control element generation engine 660 generates other types of notification messages comprising, for example, assessment or outcome prediction notifications with content, alert icons, warnings with various severity levels, etc., email messages with content and indicators showing various severity levels, short message service (SMS) messages, collaboration app messages such as a Slack® messages, Microsoft® Teams® notifications, Facebook® Workplace™ notifications, WhatsApp® messages, messages via IT Applications such as CRM systems, etc., desktop notifications, browser notifications, etc., to user devices 714 (in FIG. 7) of users 108 (in FIG. 1). The control element generation engine 660 includes content with cues and assessment and outcome level indicators.

In some embodiments, the modeling engine 650 transmits the results of procedures performed using the trained data libraries 646. In the exemplary implementations shown, the modules of the IMP subsystem 700 (600 in FIG. 6) comprise the data identification (extraction) engine 644, the modeling engine 650, the control element generation engine 660, and trained data libraries 646. In some embodiments, the data identification (extraction) engine 644, the modeling engine 650, and the control element generation engine 660 are stored in the IMP system memory 626 and executed by the processor 402 (in FIG. 4). The identification engine 644 via an internal messaging pipeline 705g (in FIG. 7) may identify any or a plurality of the elements 701 and store them in separate blocks 705a, 705b, 705c, 705d. 705e, and 705f.

In some embodiments, the IMP system 700 comprises an API system comprising an API gateway 606 coupled to data portals. As many portals as necessary may be configured to provide data streams from different locations. The API gateway 606 receives the data sets described. The API system 700 comprises the API gateway 606 and API connectors 624 with corresponding APIs “API1” 626, “API2” 628, “API3” 630, and “API4” 632 configured to connect to multiple external applications. The API system 700 accesses the external applications 701 (via data portals 602 and 604) using, for example, a representational state transfer (REST) web services architecture that uses a hypertext transfer protocol (HTTP). The system 700 uses the data-trained libraries (stored in the databases) to generate different areas, and in turn, to generate assessment or predictive models for each user (e.g., medical professional or patient).

In some embodiments, targeted, medical-related notification messages may be configured as micro-messages comprising, for example, short lines of text reciting preventive or post-procedure recommendations, providing links to access medical content, recommendations or developments etc., that aim to improve medical outcomes in patient. In some embodiments, the IMP system 700, in communication with one or more data trained libraries, dynamically generates medical content using one or more models, medical assessments, and personalized recommendations for each of the users.

In some embodiments, the medical system 100 generates target user/patient reports comprising of one or more of user actions performed in response to the targeted, contextual control elements, medical behavior patterns associated with specific users, medical behavior improvement trends, and a correlation between the medical behavior patterns and compliance based on preferred medical protocols.

In some embodiments, the IMP system 114 (FIG. 1) may include a training engine 470 that collects data on specific users, anonymizes the data and uses the data to train the neural networks. In some implementations, the data includes real-world procedural data, which is continuously provided via feedback loops.

The data model engine 650 receives the data sets on elements from the data identification (extraction) engine 644 via the internal messaging pipeline 705g. In some embodiments, the data model engine 650 performs machine learning by executing one or more of rule-based, supervised, and unsupervised machine learning algorithms on the elements using metadata 652, rules 654, and co-relations 656 to dynamically generate one or more medical procedure models for each of the users. In an embodiment, the data model engine 650 retrieves the metadata 652 comprising, for example, user profiles, medical information associated with the users, etc., from a metadata storage device for performing the computations required for dynamically generating one or more procedure assessment or prediction models for each of the users. In some embodiments, the data model engine 650 performs machine learning 708d by executing one or more of rule-based, supervised, and unsupervised machine learning algorithms on the elements using metadata 652, rules 654, and co-relations 656 to dynamically generate a medical file for each of the users.

In one embodiment, the data model 650 comprises a recommendation engine 658 for generating recommendation inputs to the control element generation engine 660 for creating recommendations to execute changes to configurations of medical procedures based on the dynamically generated models of each of the users. In some embodiments, the recommendation engine 658 of the data model 650 renders recommendation inputs for dynamically generating medical recommendations configured to change the behavior in users, for example, before a medical procedure.

In some embodiments, the databases are configured as cloud-based databases implemented in a cloud computing environment, where computing resources are delivered as a service over the network. As used herein, “cloud computing environment” refers to a processing environment comprising configurable computing physical and logical resources, for example, networks, servers, storage media, virtual machines, applications, services, etc., and data distributed over the network. The cloud computing environment provides on-demand network access to a shared pool of the configurable computing physical and logical resources. In some embodiments, one or more of the trained data libraries illustrated in FIG. 4 comprise a collection of medical scenarios that may be used by the control element generation engine 660 to automate the generation of the targeted, data-based control elements, for example, notification messages and deliver medical progress content to a user 108. In another embodiment, one or more of the trained data libraries is a medical insights library comprising a collection of critical factors and their sources. In some embodiments, one or more of the trained data libraries comprise predefined medical situations and scenarios to which different users behave differently.

The data model 650 transmits the dynamically generated medical assessment models and the recommendation inputs to the control element generation engine 660 via the internal messaging pipeline 711 (in FIG. 7). The control element generation engine 660 may dynamically generate targeted, control elements specific to a specific user 108a or 108b identified from among multiple users 108n using the dynamically generated medical outcome models, the medical procedure, and one or more trained data libraries.

The control elements of the technology dynamically renders one or more of the targeted, contextual control elements to a user device of the target user through one or more of multiple delivery channels, for example, email, SMS, a mobile app, a collaboration app, etc., via respective APIs, for providing assessments, predictions, outcomes, or the like, to guide the target user through preferred scenarios to enhance medical care and outcomes. The user device 714 (in FIG. 7) is an electronic device, for example, one or more of a personal computer, a tablet computing device, a mobile computer, a mobile phone, a smart phone, a portable computing device, a laptop, a personal digital assistant, a wearable device such as a smart glass, a smart watch, etc., a touch centric device, a workstation, a client device, a portable electronic device, a network enabled computing device, an interactive network enabled communication device, a gaming device, an image capture device, a web browser, any other suitable computing equipment, combinations of multiple pieces of computing equipment, etc. The control elements deliver data on the user device 714 of the target user 108 (FIG. 1) via the API gateway 606. In some embodiments, the control elements may automatically select one or more of the data delivery channels or data portals.

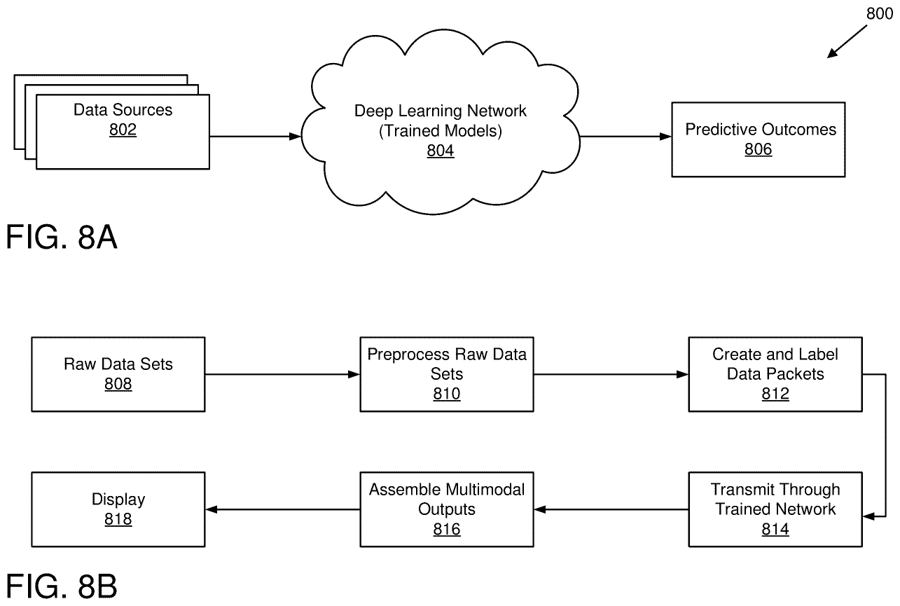

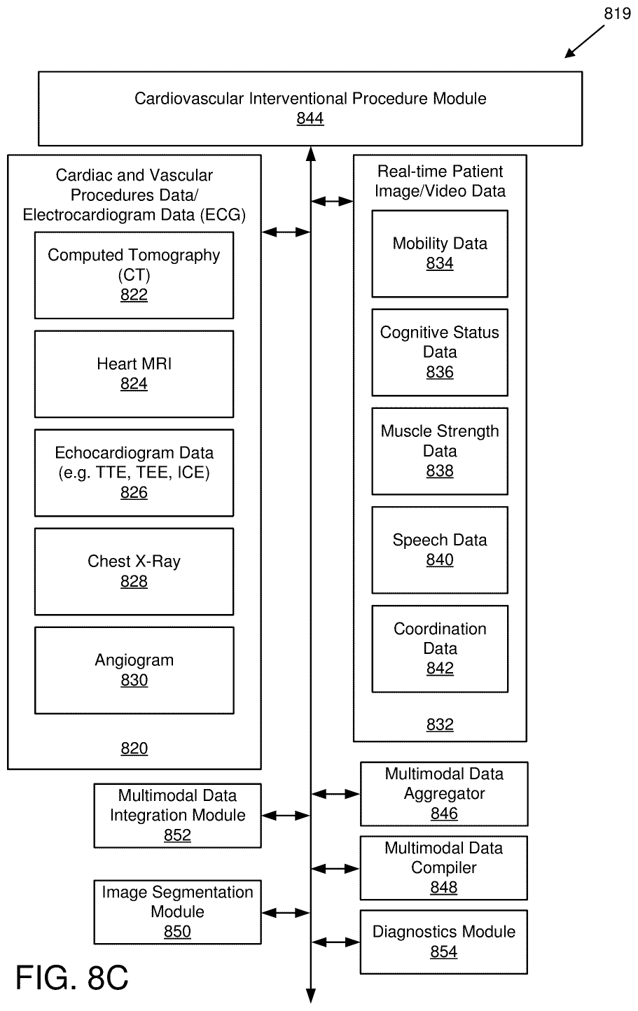

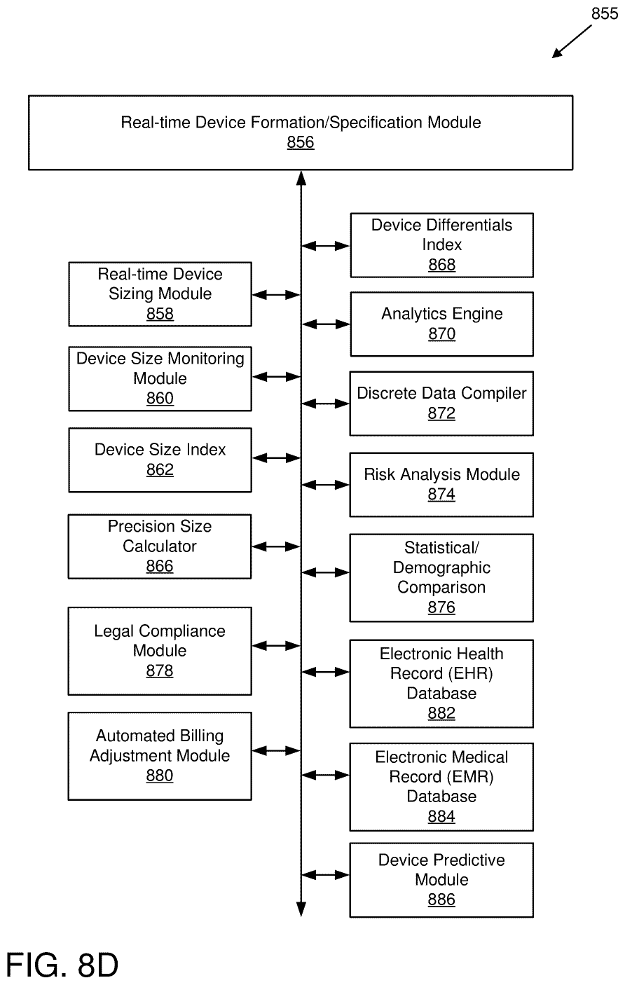

Referring now to FIG. 8A, various software modules and engines designated generally by reference numeral 800 are illustrated, which dynamically and continuously gather data, as the products of the present implementation are in use. The various software modules and engines referred to or described here may be whole programs, applications, functions, logic, software libraries, classes or code in an object-oriented language. The AI model of the present implementation uses diverse medical data sets. A rich set of features are used to identify multimodal correlations between variables to predict medical outcomes for medical professionals. With artificial intelligence, the system identifies patterns that are not easily evident to humans, thereby eliminating the clinical and population biases that can occur. These various software modules and engines include multiple data sources 802 configured to compile data (obtained via data collection portals), which data is transmitted through deep learning networks (trained models) 804 to produce predictive outcomes 806.

Referring now to FIG. 8B, raw data sets, as illustrated by block 808, are collected and the raw data sets are preprocessed, as illustrated by block 810. After preprocessing, the raw data is labeled and assembled into packets, as illustrated by block 812. The assembled data is transmitted through trained networks, as illustrated by block 814. Multimodal outputs are assembled, as illustrated by block 816, which outputs are transmitted to identified destinations and displayed, as illustrated by block 818.

Referring to FIG. 8C, the IMP platform 114 (in FIG. 1), as illustrated generally by reference numeral 819, illustrates the processes by a cardiovascular interventional procedure module 844. The various hardware and software components include cardiac and vascular procedures data and electrocardiogram data (ECG) designated by block 820. Examples of the data include, but are not limited to, computed tomography (CT) as designated by block 822, heart MRI as designated by block 824, echocardiogram data (e.g., TTE, TEE, and ICE) as designated by block 826, chest X-ray data as designated by block 828, angiogram data as designated by block 830. The real-time patient image/video data designated by block 832, may include mobility data in block 834, cognitive status data in block 836, muscle strength data in block 838, speech data in block 840, and coordination data in block 842. Additional software modules include multimodal data integration module in block 852, image segmentation module in block 850, multimodal data aggregator in block 846, multimodal data compiler in block 848, and diagnostics module in block 854.

In some embodiments, the various types of data are recorded during clinical visits. For example, as typical, a cardiologist when evaluating a patient's medical condition, would request tests that would determine the various disparate data. Real-time patient image and video data 832 includes visual data to inform on the patient's condition. For example, the mobility data 834 may reflect a specific patient's gait, whether steady or unbalanced. The cognitive status data 836 reflects cognitive decline in a patient who may have suffered one or more heart attacks. As another example, deteriorating muscle strength is an indicator of cardiovascular disease. As yet another example, speech data 840 is critical, as heart failure is a major global health concern, increasing in prevalence, and affects the larynx and breathing, and therefore, the quality of speech. Coordination data 842 is another important indicator of declining cardiovascular health. Gathering all this data in real time enables the IMP platform 114 to aggregate the multimodal data as illustrated in block 846, and compile segments of the data, as illustrated in block 848. Images may be segmented by the image segmentation module 850 to create composite multimodal images and the multimodal data integration module 852 is configured to integrate data segments to external or internal application as needed to route them to identified destinations. The multimodal data is processed by the diagnostics module 854 to indicate diagnostic frameworks for either groups of patients or specific patients.

Referring now to FIG. 8D, in some embodiments, the IMP platform 114 has real-time device formation and specification provision processes. The real-time device formation and specification module 856 gathers, evaluates, and provides precise measurements, which data is used to prescribe devices that are designed for each specific patient use in real time. Output data from medical procedures may also be provided to device vendors to enable precise manufacturing benefits. The system and processes designated generally by reference numeral 855 comprises a real-time device sizing module 858, device size monitoring module 860, a device size index 862, a precision size calculator 866, a device differentials index 868, an analytics engine 870, a discreate data compiler 872, a risk analysis module 874, a statistical/demographic comparison 876, a Legal (e.g, FDA, HIPPA, ISO, GDPR, SOC 2, etc.) compliance module 878, an automated billing adjustment module 880, an electronic health record (EHR) database 882, an electronic medical record (EMR) database 884, and a device predictive module 886.

When a specific patient's cardiac valve (e.g., the aortic or the mitralvalve) is badly deteriorated and beyond repair and other treatments are not an option, replacing them with a prosthetic valve can be necessary. To replace a heart valve, a surgeon removes the heart valve and replace it with a mechanical valve (also known as a metal valve) or biological valves (or bioprosthesis) including a valve made from cow, pig, or human heart tissue (homograft valves). A special tool, known as a heart valve sizer, is used to determine the size (diameter, surface area) of the heart valve replacement. This unique device has multiple attachments that are lowered into the heart until a match for a specific patient is found. In accordance with the present implementation, processing multimodal real time data provides a distinct approach to sizing a device before placement in a specific patient. With training, the neural network systems described here would accurately size the devices in real time.

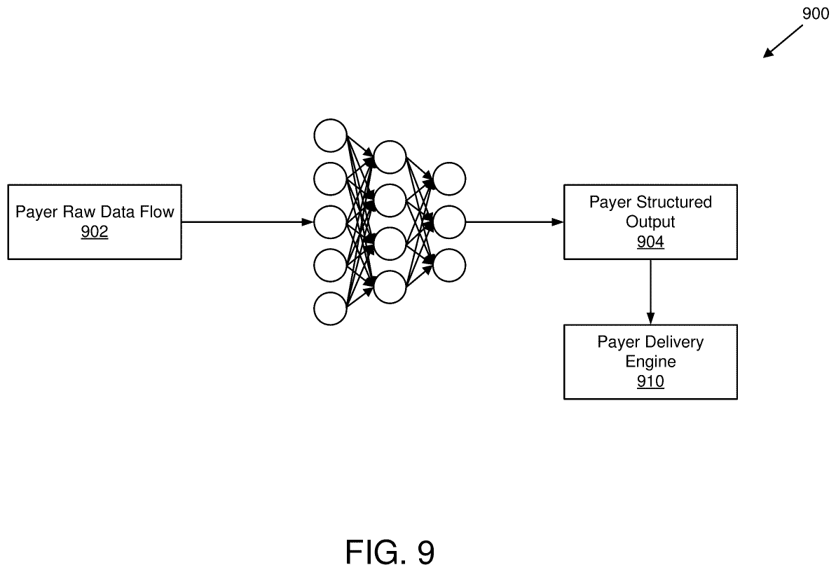

Referring now to FIG. 9, the artificial intelligence system illustrated generally by reference numeral 900 comprises various components implemented as software modules or engines including payer raw data flow module 902, an output from which flows into neural networks designated by reference numeral 903, with an output from the neural networks 903 providing payer structured output 904. The neural networks 903 are trained to structure the raw data imported or collected from different payer sources to structure the payer data, which structured data is delivered to a payer delivery engine 810. A feedback loop from the module 904 may lead back (iterate) to the neural networks 903. The module 902 is configured to continuously and dynamically capture raw data from all sources.

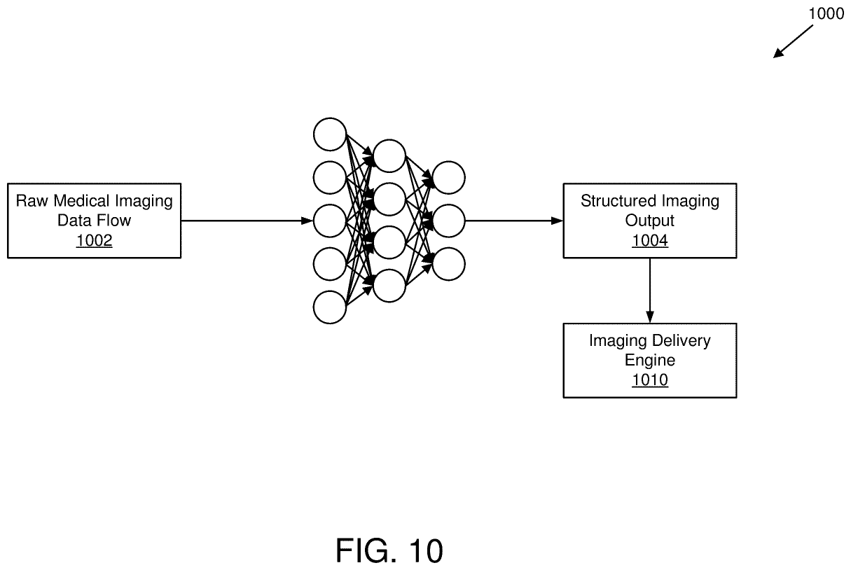

Referring now to FIG. 10, the artificial intelligence system illustrated generally by reference numeral 1000 comprises various components implemented as software modules or engines including payer raw data flow module 902, an output from which flows into neural networks designated by reference numeral 903, with an output from the neural networks 903 providing payer structured output 904. The neural networks 903 are trained to structure the raw data imported or collected from different payer sources to structure the payer data, which structured data is delivered to a payer delivery engine 810. A feedback loop from the module 904 may lead back (iterate) to the neural networks 903. The module 902 is configured to continuously and dynamically capture raw data from all sources

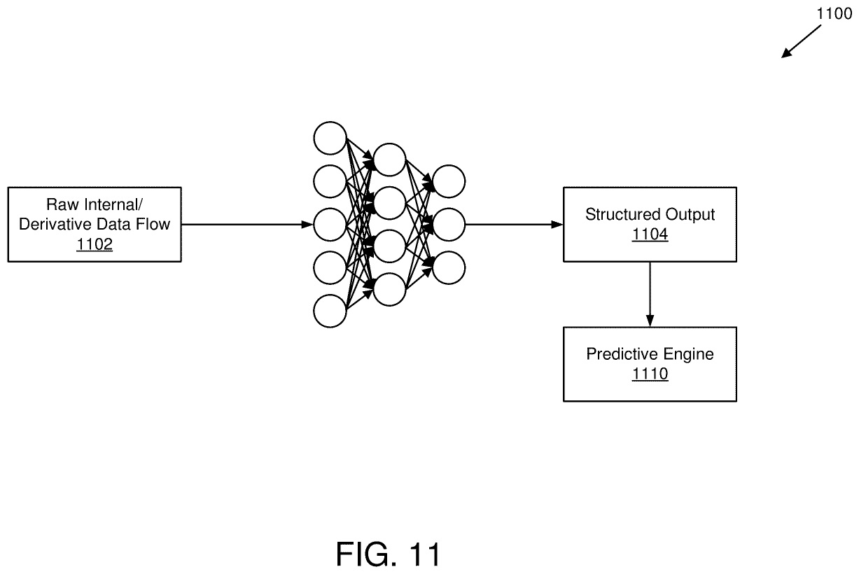

Referring now to FIG. 11, the artificial intelligence system illustrated generally by reference numeral 1100 comprises various components implemented as software modules or engines including raw internal/derivative data flow module 1102, an output from which flows into neural networks designated by reference numeral 1103, with an output from the neural networks 1103 providing structured output 1104. The neural networks 1103 are trained to structure the raw data imported or collected from different payer sources to structure the payer data, which structured data is delivered to a predictive engine 1110. A feedback loop from the module 904 may lead back (iterate) to the neural networks 903. The module 1102 is configured to continuously and dynamically capture raw data from all sources.

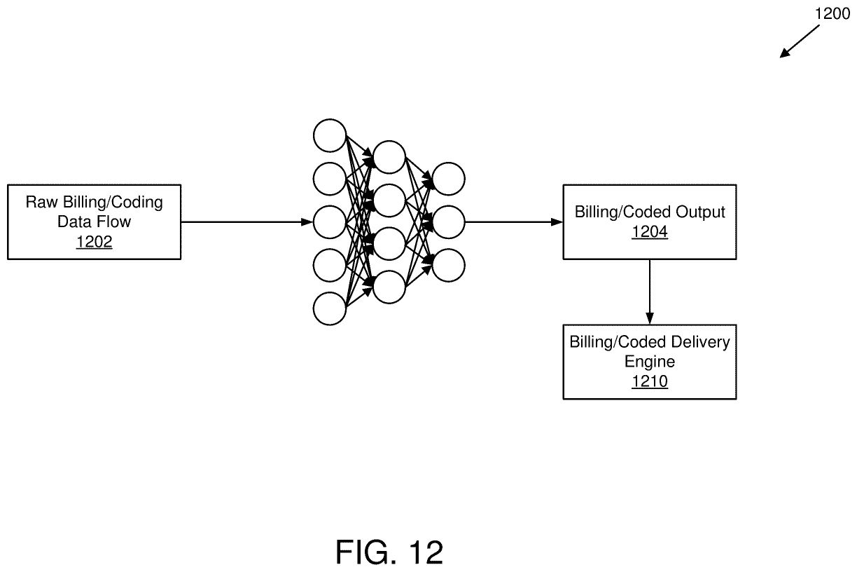

Referring now to FIG. 12, the artificial intelligence system illustrated generally by reference numeral 1200 comprises various components implemented as software modules or engines including raw billing/coding data flow module 1202, an output from which flows into neural networks designated by reference numeral 1203, with an output from the neural networks 1203 providing billing/coded output 1204. The neural networks 1203 are trained to structure the raw data imported or collected from different payer sources to structure the payer data, which structured data is delivered to a billing/coding delivery engine 1210. A feedback loop from the module 1204 may lead back (iterate) to the neural networks 1203. The module 1202 is configured to continuously and dynamically capture raw data from all sources.

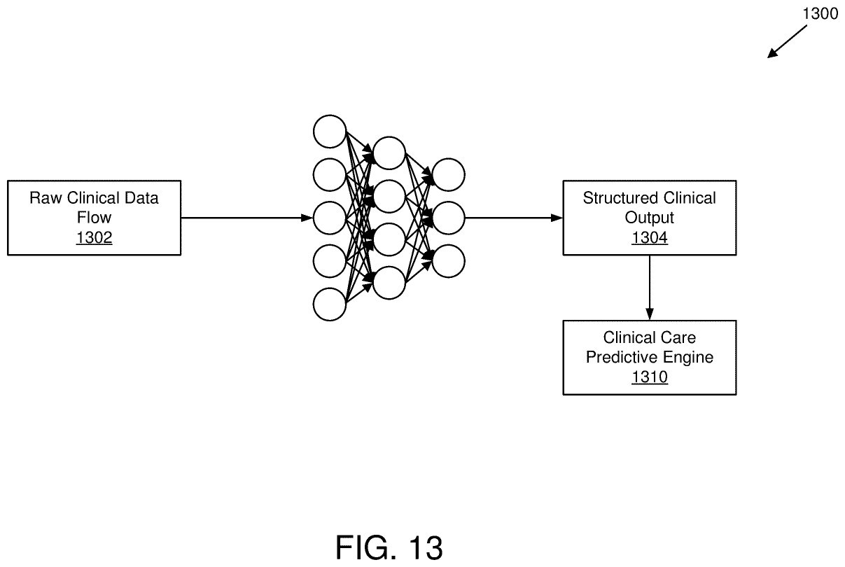

Referring now to FIG. 13, the artificial intelligence system illustrated generally by reference numeral 1300 comprises various components implemented as software modules or engines including raw clinical data flow module 1302, an output from which flows into neural networks designated by reference numeral 1303, with an output from the neural networks 1303 providing structured clinical output 1304. The neural networks 1303 are trained to structure the raw data imported or collected from different payer sources to structure the payer data, which structured data is delivered to a clinical care predictive engine 1310. A feedback loop from the module 1304 may lead back (iterate) to the neural networks 1303. The module 1302 is configured to continuously and dynamically capture raw data from all sources.

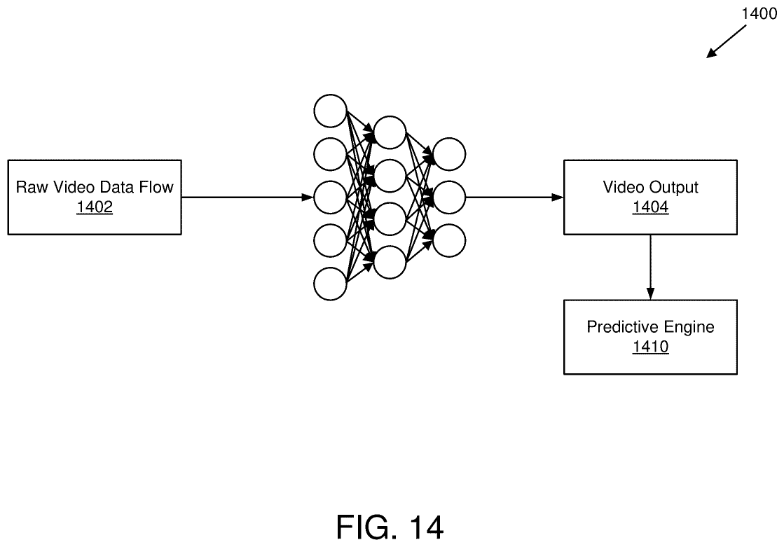

Referring now to FIG. 14, the artificial intelligence system illustrated generally by reference numeral 1400 comprises various components implemented as software modules or engines including raw video data flow module 1402, an output from which flows into neural networks designated by reference numeral 1403, with an output from the neural networks 1403 providing video output 1404. The neural networks 1403 are trained to structure the raw data imported or collected from different payer sources to structure the payer data, which structured data is delivered to a predictive engine 1410. A feedback loop from the module 1404 may lead back (iterate) to the neural networks 1403. The module 1402 is configured to continuously and dynamically capture raw data from all sources.

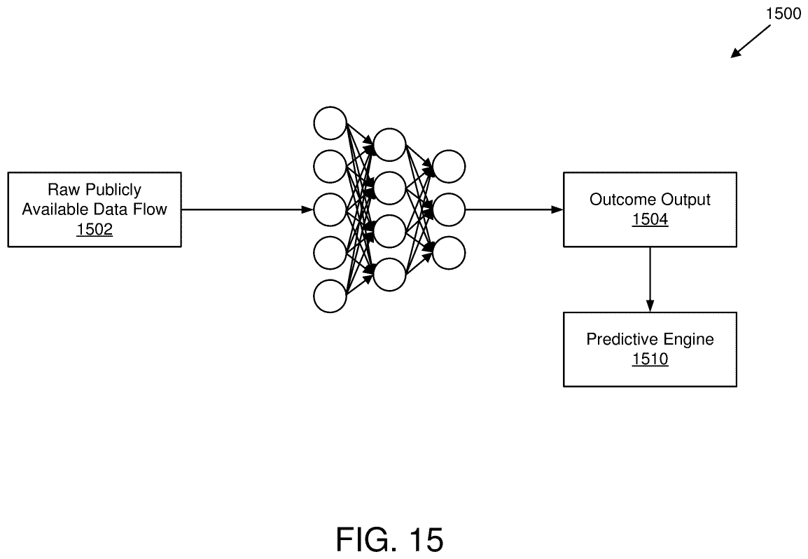

Referring now to FIG. 15, the artificial intelligence system illustrated generally by reference numeral 1500 comprises various components implemented as software modules or engines including raw publicly available data flow module 1502, an output from which flows into neural networks designated by reference numeral 1503, with an output from the neural networks 1503 providing payer outcome output 1504. The neural networks 1503 are trained to structure the raw data imported or collected from different payer sources to structure the payer data, which structured data is delivered to a payer delivery engine 1510. A feedback loop from the module 1504 may lead back (iterate) to the neural networks 1503. The module 1502 is configured to continuously and dynamically capture raw data from all sources.

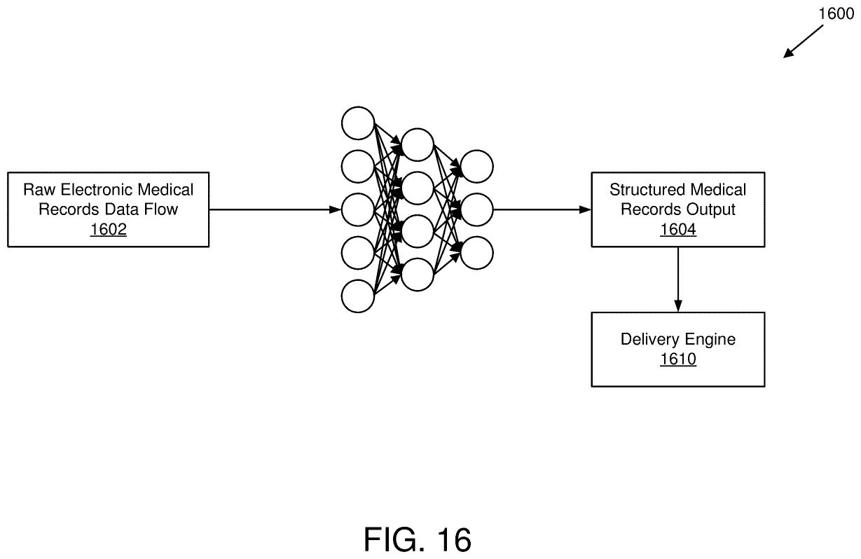

Referring now to FIG. 16, the artificial intelligence system illustrated generally by reference numeral 1600 comprises various components implemented as software modules or engines including raw electronic medical records data flow module 1602, an output from which flows into neural networks designated by reference numeral 1603, with an output from the neural networks 1603 providing payer structured medical records output 1604. The neural networks 1603 are trained to structure the raw data imported or collected from different payer sources to structure the payer data, which structured data is delivered to a payer delivery engine 1610. A feedback loop from the module 1604 may lead back (iterate) to the neural networks 1603. The module 1602 is configured to continuously and dynamically capture raw data from all sources.

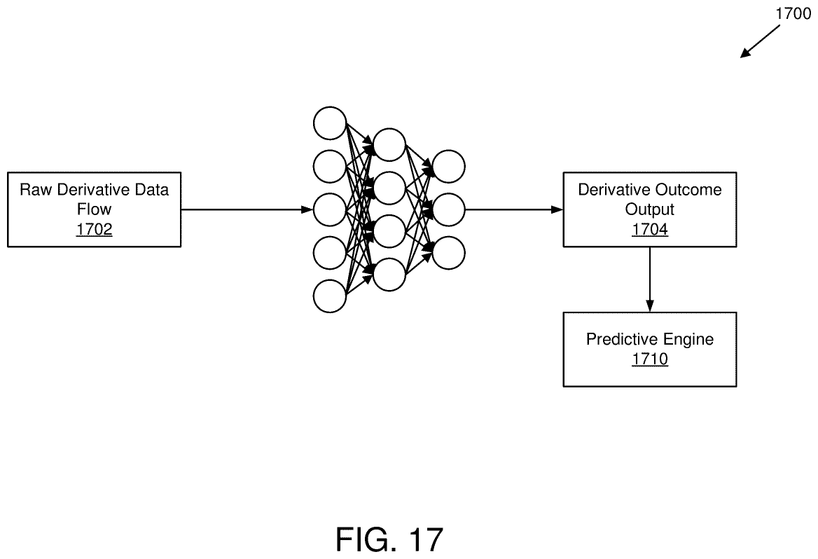

Referring now to FIG. 17, the artificial intelligence system illustrated generally by reference numeral 1000 comprises various components implemented as software modules or engines including raw derivative data flow module 1702, an output from which flows into neural networks designated by reference numeral 1703, with an output from the neural networks 1703 providing derivative outcome output 1704. The neural networks 1703 are trained to structure the raw data imported or collected from different payer sources to structure the payer data, which structured data is delivered to a predictive engine 1710. A feedback loop from the module 1704 may lead back (iterate) to the neural networks 1703. The module 1702 is configured to continuously and dynamically capture raw data from all sources.

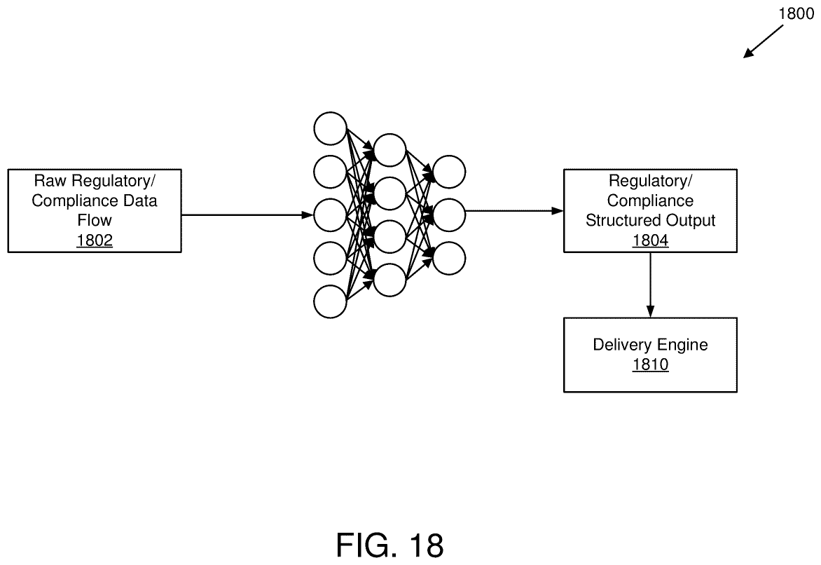

Referring now to FIG. 18, the artificial intelligence system illustrated generally by reference numeral 1800 comprises various components implemented as software modules or engines including raw regulatory/compliance data flow module 1802, an output from which flows into neural networks designated by reference numeral 1803, with an output from the neural networks 1803 providing regulatory/compliance structured output 1804. The neural networks 1803 are trained to structure the raw data imported or collected from different payer sources to structure the payer data, which structured data is delivered to a delivery engine 1810. A feedback loop from the module 1804 may lead back (iterate) to the neural networks 1803. The module 1802 is configured to continuously and dynamically capture raw data from all sources.

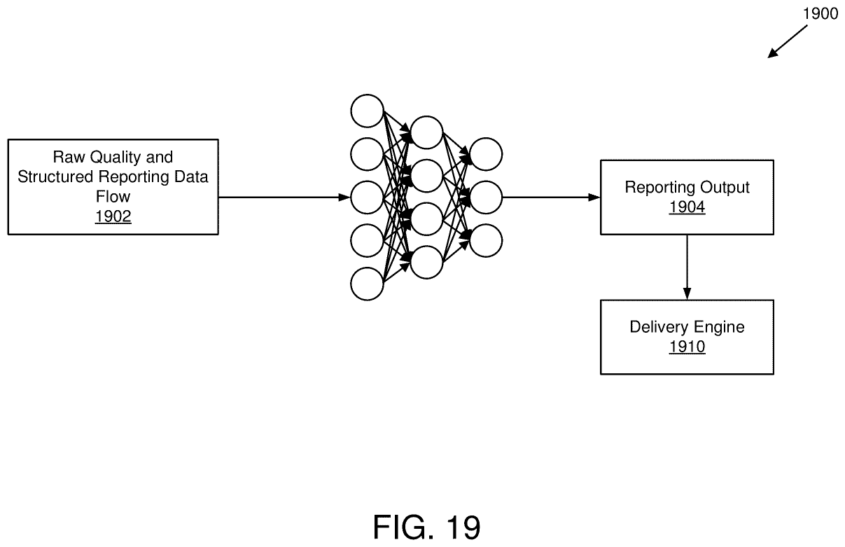

Referring now to FIG. 19, the artificial intelligence system illustrated generally by reference numeral 1900 comprises various components implemented as software modules or engines including raw quality and structured reporting data flow module 1902, an output from which flows into neural networks designated by reference numeral 1903, with an output from the neural networks 1903 providing reporting output 1904. The neural networks 1903 are trained to structure the raw data imported or collected from different payer sources to structure the payer data, which structured data is delivered to a delivery engine 1910. A feedback loop from the module 1904 may lead back (iterate) to the neural networks 1903. The module 1902 is configured to continuously and dynamically capture raw data from all sources.

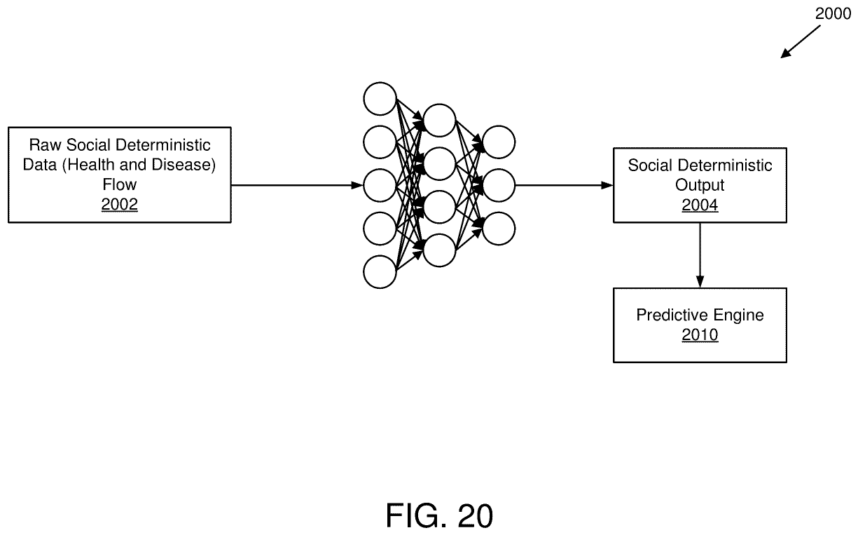

Referring now to FIG. 20, the artificial intelligence system illustrated generally by reference numeral 1000 comprises various components implemented as software modules or engines including raw social deterministic data (health and disease) flow module 2002, an output from which flows into neural networks designated by reference numeral 2003, with an output from the neural networks 2003 providing payer social deterministic output 2004. The neural networks 2003 are trained to structure the raw data imported or collected from different payer sources to structure the payer data, which structured data is delivered to a predictive engine 2010. A feedback loop from the module 2004 may lead back (iterate) to the neural networks 2003. The module 2002 is configured to continuously and dynamically capture raw data from all sources.

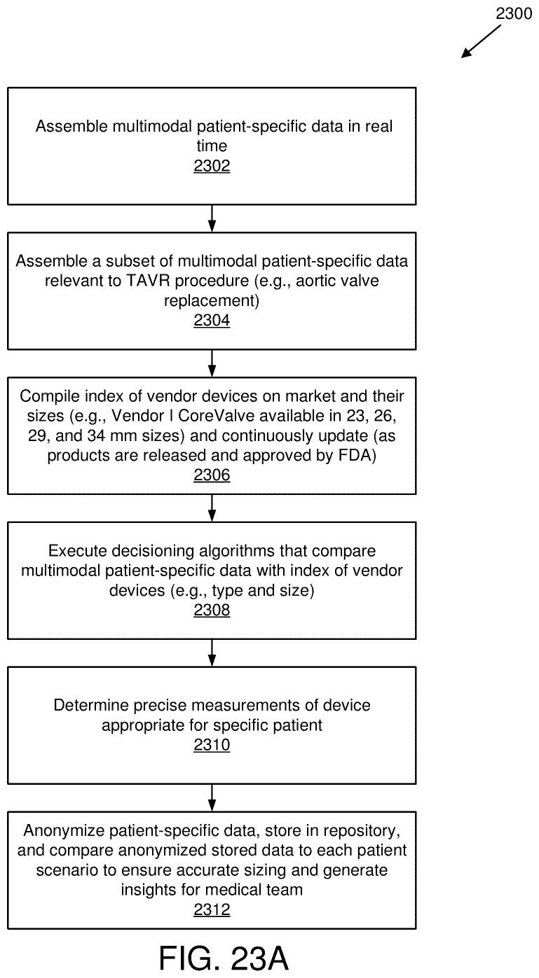

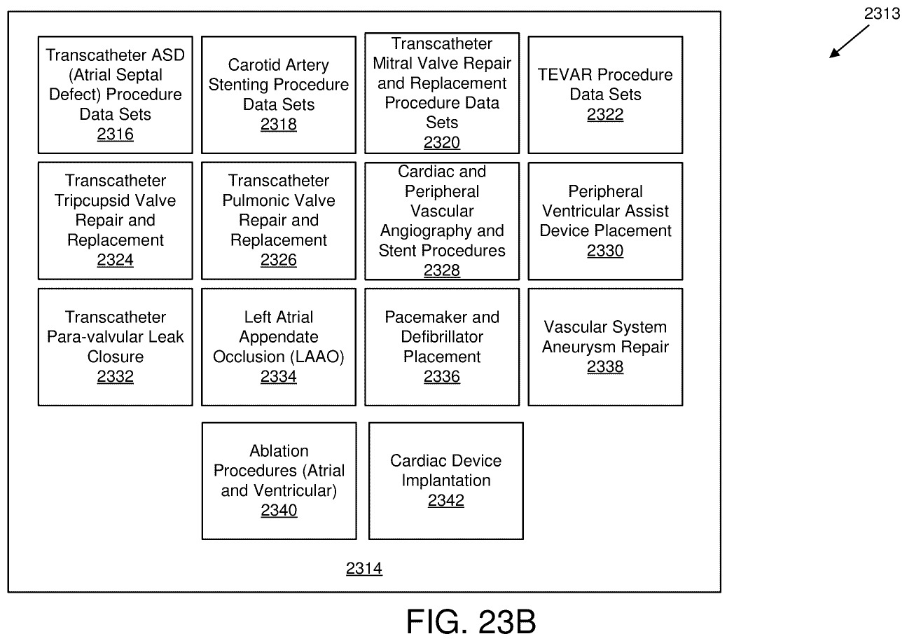

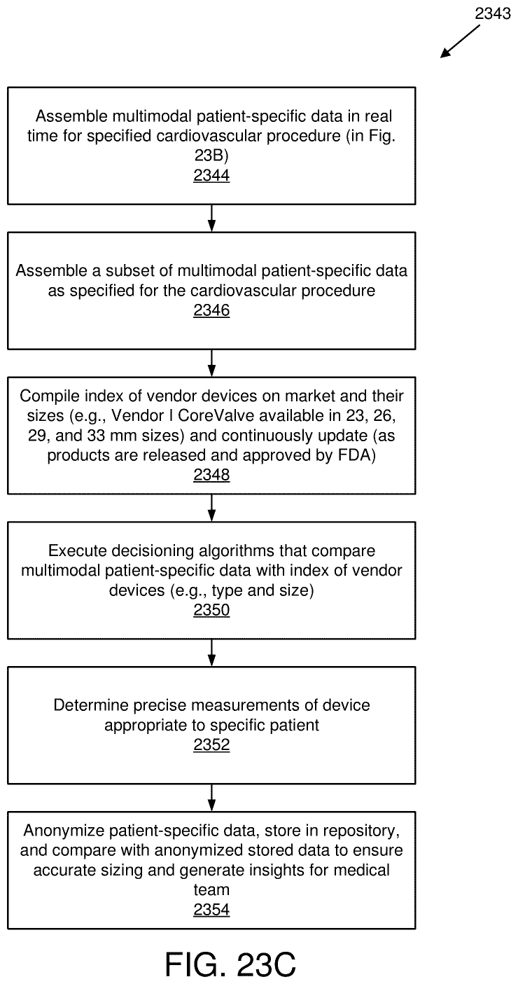

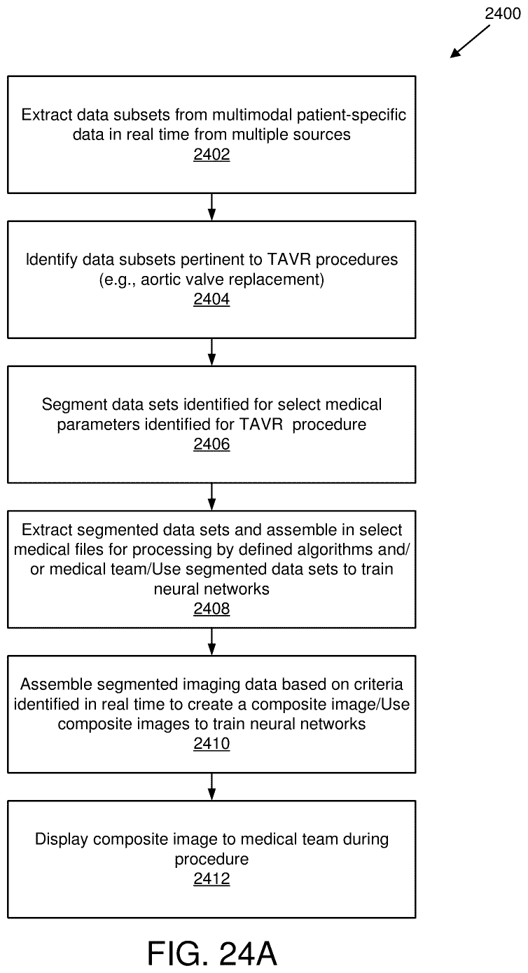

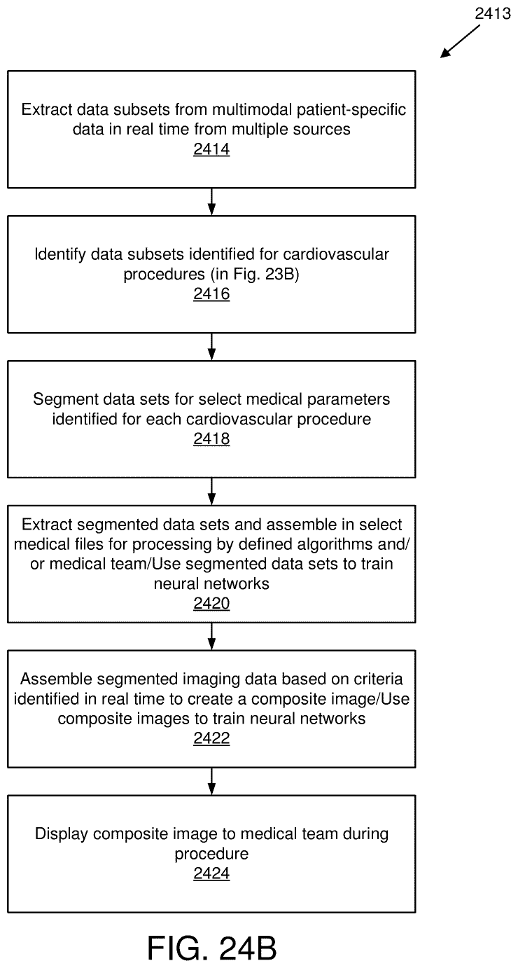

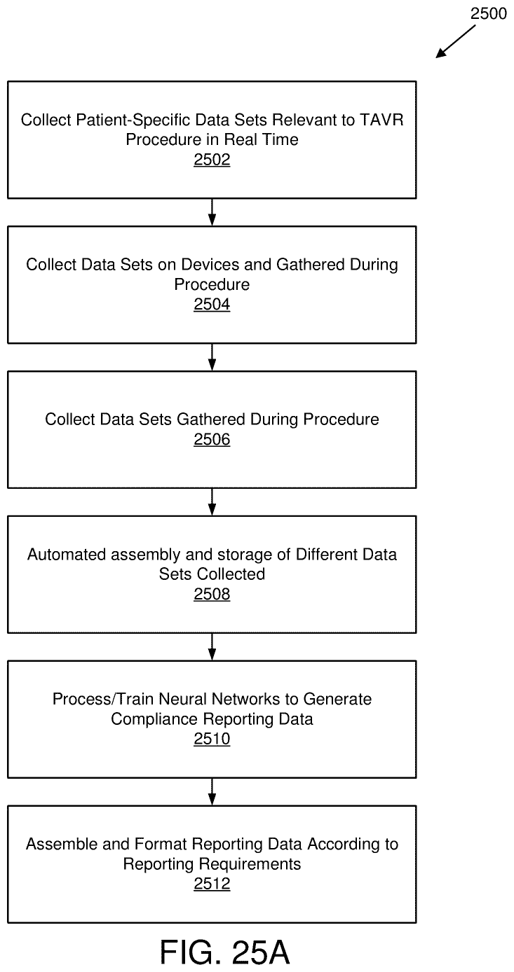

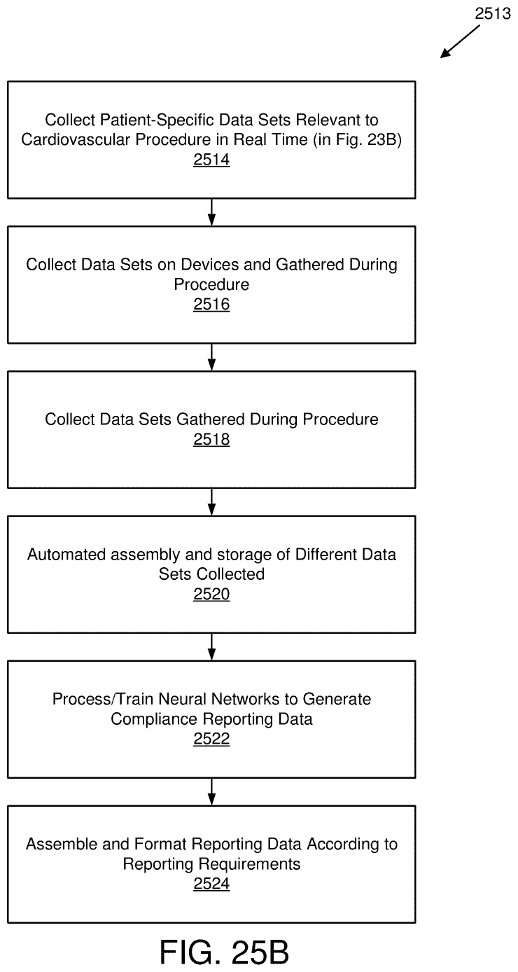

Referring now to FIG. 23, the machine learning systems of the present implementation are illustrated generally by reference numeral 2100. In accordance with some embodiments, the machine learning systems in accordance with the present implementation are configured in distributed layers. In some embodiments the micro machine learning system 2106 has a plurality of integrated layers (as illustrated in FIGS. 9 through 20) coupled to multiple data sources and neural networks that are trained to provide insights and predict outcomes. In some embodiments the regional machine learning system 2104 has a plurality of integrated layers (as illustrated in FIGS. 9 through 20) coupled to multiple data sources and neural networks that are trained to provide insights and predict outcomes. In some embodiments the global machine learning system 2102 has a plurality of integrated layers (as illustrated in FIGS. 9 through 20) coupled to multiple data sources and neural networks that are trained to provide insights and predict outcomes.

The present technology also relates to an apparatus or system for performing the operations described. This apparatus or system may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CDROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

It should be recognized that components of the present technology can take the form of an entirely hardware embodiment, an entirely software embodiment or an implementation containing both hardware and software elements. In some implementations, this technology is implemented in software, which includes but is not limited to, firmware, resident software, microcode, etc. Furthermore, this technology can take the form of a computer program product accessible from a computer-usable or computer-readable medium providing program code for use by or in connection with a computer or any instruction execution system. For the purposes of this description, a computer-usable or computer-readable medium can be any apparatus that can contain, store, communicate, propagate, or transport the program for use by or in connection with the instruction execution system, apparatus, or device.

Finally, the algorithms and graphical displays presented here are not inherently related to any particular computer or other apparatus. Various general-purpose systems may be used with programs in accordance with the teachings herein, or it may prove convenient to construct more specialized apparatuses to perform the required method steps. The required structure for a variety of these systems will appear from the description below. In addition, the present implementation is not described with reference to any particular programming language. It will be appreciated that a variety of programming languages may be used to implement the teachings of the implementation as described herein.

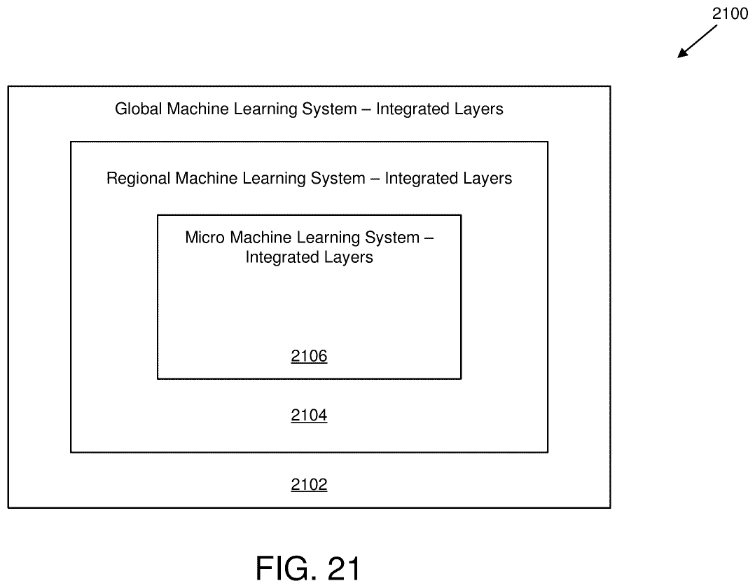

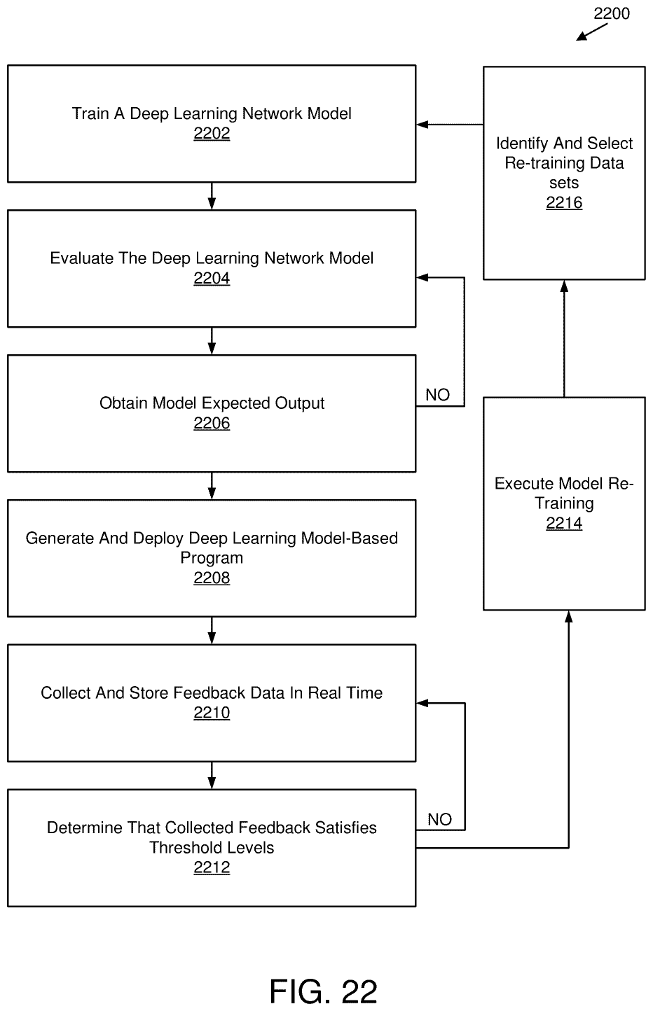

Referring now to FIG. 22, the process for training the deep learning network models is illustrated generally at 2200. Examples of the deep learning network models are illustrated in FIGS. 9-20 and integrated layers are illustrated in FIG. 21. The method begins at block 2202, which includes one or more functions and operations for training a deep learning network model 2202 with the data sets described above for example in FIGS. 2 and 3. The process 2200 proceeds to the next block 2204, including one or more functions and operations for evaluating the deep learning network model. The process 2200 proceeds to the next block 2206, including one or more operations for obtaining the expected outputs from the model. The process 2200 proceeds to the next block 2208, including one or more functions and operations for generating and deploying deep learning model-based programs. The process 2200 proceeds to the next block 2210, including one of more functions and operations for collecting and storing feedback data in real time. The process 2200 proceeds to the next block 2212, including one or more operations for determining that the collected feedback satisfies threshold levels that are either predefined or set. The process 2200 proceeds to the next block 2214, including one or more functions or operations for executing re-training of the models. The process 2200 proceeds to the next block 2216, including one or more functions and operations for identifying and selecting the re-training data sets. The process 2200 proceeds to the block 2202.

In operation, in some embodiments, the distributed learning architecture of the deep learning network model may include three interconnected layers including a back-end integration layer, an AI orchestration layer, and a front-end interaction/visualization layer. The back-end integration layer may be coupled to the AI orchestration layer via one or more back-end interfaces/gateways, and the front-end interaction/visualization layer may be coupled to the AI orchestration layer via one or more front-end interfaces/gateways.

The back-end integration layer may comprise various disparate data sources and systems that provide data and/or services associated with the medical domain in which the IMP platform 114 is used to facilitate integrating AI informatics. For example, for medical procedures, the back-end integration layer may comprise various disparate healthcare data sources and healthcare systems that provide healthcare data and/or services associated with generating or consuming the healthcare data. The back-end integration layer may comprise one or more internal data sources/systems, as well as one or more external data sources/systems that provide input data used that may be used for teaching and training one or more AI models directed domain/industry specific solutions.

For example, as applied to healthcare including medical procedures, the one or more internal data sources/systems, as well as one or more external data sources/systems that provide input data that may be used for teaching and training one or more AI models to provide various healthcare solutions, are referred to herein generally as healthcare AI models. In this regard, the one or more internal and/or external data sources/systems may include medical imaging systems configured to provide medical image data, patient records systems configured to provide patient health information, medical reporting systems configured to provide patient pathology reports, and the like. The back-end integration layer may also comprise healthcare reporting/monitoring systems that provide real-time (or substantially real-time) contextual input information associated with patients, clinicians, and medical supplies/instruments at one or more healthcare facilities (e.g., physiological information, location information, workflow information, etc.) that may be used as input when the healthcare models are operated in association with performance of actual clinical and/or operational workflows in real-time to enhance performance of these workflows with AI oversight.

Various embodiments, the data elements and training data included in the back-end integration layer may be configured to be searchable and may be accessed by multiple systems for building new models and algorithms.

Referring now to FIG. 23A, the process 2300 for assembling multimodal data sets for performing a procedure is described. The process 2300 for assembling the multimodal data is described for performing a aortic valve replacement (“TAVR”) procedure. The process 2300 begins at block 2302, which includes one or more functions or operations for assembling multimodal patient-specific data in real time. The data may be collected, sorted, and labeled into data sets. The process 2300 proceeds to the next block 2304, including one or more functions or operations for assembling a subset of multimodal patient-specific data relevant to the aortic valve replacement TAVR procedure. Th process 2300 proceeds to the next block 2306, which includes one or more functions or operations for compiling an index of vendor devices available on the market and their sizes. As one example, the Vendor I CoreValve provides devices in various sizes, such as 23 mm, 26 mm, 29 mm, and 34 mm. The index is continuously and dynamically updated as products are released and approved by the Food and Drug Authority (“FDA”). The process 2300 proceeds to the next block 2308, which includes one or more functions and operations for executing decisioning algorithms that compare the multimodal patient-specific data with the index of vendor devices (e.g., type and size). The process 2300 proceeds to the next block 2310, which includes one or more functions and operations for determining precise measurements of a device appropriate for a specific patient. The process 2300 proceeds to the next block 2312 including one or more functions and operations for anonymizing patient-specific data and storing the anonymized data in one or more repositories. The anonymized data is compared to each instance of a patient scenario to ensure accurate sizing and generate insights for the medical team, before or during the medical procedure. In some instances, a simulation of a likely outcome may be generated for the medical team before the procedure is initiated.

Referring now to FIG. 23B, a database 2313 of multiple procedures stores templates and data sets relevant to the multiple procedures. In some embodiments, the database 2313 is configured to include templates and data sets for a transcatheter ASD (atrial septal defect) procedure, illustrated by block 2316. The atrial septal defect (ASD) transcatheter repair is a procedure to fix a hole in the atrial septum. As recognized by those skilled in the medical field, the atrial septum is a wall that separates the right and left upper chambers in the heart (atria). This hole is referred to as an atrial septal defect or ASD. A medical professional determines that this defect is present, upon observing that the blood flow is abnormal, from the left atrium into the right atrium. This abnormal blood flow typically causes the heart to pump extra blood out into the lungs, which may damage the lung blood vessels if left untreated. Typically, this condition, also results in the right-side pumping chamber (right ventricle) becoming enlarged. As a result, it has to pump harder than it should to get blood out into the lungs. The ASD transcatheter repair requires a long, flexible tube (a catheter) and a small device to close this hole. An interventional cardiologist inserts the catheter through a blood vessel in the groin. Inside the catheter is a small device folded up like an umbrella. The cardiologist moves the catheter all the way to the heart's septum. The small device comes out of the tube and plugs up the hole in the atrial septum, at which point the cardiologist removes the catheter from the body. Over time, tissue grows over the device and holds it even more firmly in place. This procedure is typically performed by inserting a hollow tube (catheter) through the groin or neck veins.

In some embodiments, the database 2313 is configured to include templates and datasets for a carotid artery stenting procedure, as illustrated by block 2318. As recognized by those skilled in the medical fields, stenting involves placing a small metal coil (stent) in a clogged artery of a patient. The stent helps prop the artery open and decreases the chance of it narrowing again. Carotid angioplasty and stenting may be used when traditional carotid surgery (carotid endarterectomy) is not possible or if medical professionals determine it is too risky.

In some embodiments, the database 2313 is configured to include templates and datasets for a transcatheter mitral valve repair and replacement procedure, as illustrated by block 2320. As recognized by those skilled in the medical fields, TMVR is a minimally invasive structural heart disease treatment to replace a damaged mitral valve without open-heart surgery. A flexible, hollow tube (catheter) is inserted through a blood vessel to reach the heart and replace or repair the mitral valve.

In some embodiments, the database 2313 is configured to include templates and datasets for a TEVAR procedure, as illustrated by block 2322. As recognized by those skilled in the medical arts, thoracic endovascular aortic repair (TEVAR) is a procedure to treat an aneurysm in the upper part of the aorta. TEVAR is a minimally invasive surgery and is performed by making a small cut (incision). With TEVAR, a device called a “stent graft” is used to reinforce the aneurysm. A stent graft is a metal tube covered in fabric that prevents the aneurysm from bursting.

In some embodiments, the database 2313 is configured to include templates and datasets for a transcatheter tricuspid valve repair and replacement procedure, as illustrated by block 2324. As is recognized by those skilled in the medical fields, transcatheter heart valve therapies are minimally invasive procedures performed to repair a diseased heart valve that involves threading a catheter (tube) through a small incision made in the skin. The device typically used to repair the diseased valve is placed in the catheter and fed through a vein in the body until it reaches the diseased heart valve. Transcatheter heart valve therapies offer some patients an alternative to open heart valve surgery. These minimally invasive transcatheter therapies allow the procedure to be performed while the patient's heart is still beating, eliminating the need for a “bypass” machine and its associated risks. Due to the minimally invasive nature of transcatheter procedures, patients recover faster and experience an improvement in symptoms soon after the procedure is completed.

In some embodiments, the database 2313 is configured to include templates and datasets for a transcatheter pulmonic valve repair and replacement procedure, as illustrated by block 2326. As is recognized by those skilled in the medical fields, transcatheter pulmonary valve replacement, also known as TPVR, is a minimally invasive structural heart disease treatment to replace a failing prosthetic pulmonary valve. The pulmonary valve controls blood flow from the heart's lower right heart chamber (ventricle) into the lungs. Severe problems with the pulmonary valve can slow blood flow or lead to right-sided heart failure. In TPVR, an interventional cardiologist replaces a damaged prosthetic pulmonary valve with a new one. They use a thin, flexible tube (catheter) to implant a new pulmonary valve without opening the chest.

In some embodiments, the database 2313 is configured to include templates and datasets for cardiac device implantation procedures, as illustrated by block 2328. As is recognized by those skilled in the medical fields, cardiac device implantation is the medical process of inserting a device under the skin of a specific patient's chest, usually below the collarbone, to treat heart conditions. The procedure is typically performed under local anesthetic, and involves making a small incision, connecting the device's leads to the heart, and testing the device.

In some embodiments, the database 2313 is configured to include templates and datasets for a peripheral ventricular assist device placement procedure, as illustrated by block 2330. As is recognized by those skilled in the medical fields, a ventricular assist device (VAD) is a device that helps pump blood from the lower chambers of the heart to the rest of the body. It is a treatment prescribed for a weakened heart or heart failure. A VAD may be used to help the heart work while waiting for other treatments, such as a heart transplant. Sometimes a VAD is used to permanently help the heart pump blood.

In some embodiments, the database 2313 is configured to include templates and datasets for a transcatheter para-valvular leak closure procedure, as illustrated by block 2332. As is recognized by those skilled in the medical fields, a paravalvular leak, also called paravalvular regurgitation, refers to a leak caused by a space left between natural heart tissue and the valve replacement from a previous transcatheter aortic or mitral valve replacement. This condition most often affects the mitral valve, rather than the aortic valve. Paravalvular leak closure is a nonsurgical procedure performed by an interventional cardiologist to repair these heart valve leaks. The procedure uses a closure device as a “plug” to block the source of the leak, guided to the appropriate valve through the femoral artery in the thigh.

In some embodiments, the database 2313 is configured to include templates and datasets for a left atrial appendage occlusion (LAAO) procedure, as illustrated by block 2334. As is recognized by those skilled in the medical fields, a left atrial appendage closure is a surgical or minimally invasive procedure to seal off the left atrial appendage (LAA), which is a small sac in the muscle wall of the left atrium (top left chamber of the heart). Removing it or closing it off can reduce the risk of stroke and eliminate the need to take blood-thinning medication.

In some embodiments, the database 2313 is configured to include templates and datasets for a pacemaker and defibrillator placement procedure, as illustrated by block 2336. As is recognized by those skilled in the medical fields, an implantable cardioverter defibrillator (ICD) is a small electronic device connected to the heart. It is used to continuously monitor and help regulate potentially fast and life-threatening electrical problems with the heart. A transvenous or “traditional” ICD, about the size of a stopwatch, is implanted under the skin just below the collarbone. The device consists of a pulse generator and wires, called leads. The pulse generator contains the battery and a tiny computer. One or more lead wires connect the pulse generator to specific locations in the heart. A pacemaker insertion is the implantation of a small electronic device that is usually placed in the chest (just below the collarbone) to help regulate slow electrical problems with the heart. A pacemaker may be recommended to ensure that the heartbeat does not slow to a dangerously low rate.

In some embodiments, the database 2313 is configured to include templates and datasets for a vascular system aneurysm repair procedure, as illustrated by block 2338. As is recognized by those skilled in the medical fields, Vascular surgeons can repair aneurysms using endovascular aneurysm repair (EVAR), which is a minimally invasive procedure that involves making two small incisions in the groin or arm. The surgeon threads a catheter through a blood vessel to the aneurysm, and places a fabric tube (endograft stent) inside the aneurysm to relieve pressure and prevent rupture. EVAR has less pain, faster healing, and improved outcomes compared to open surgery.

In some embodiments, the database 2313 is configured to include templates and datasets for ablation (atrial and ventricular) procedures, as illustrated by block 2340. As is recognized by those skilled in the medical fields, cardiac ablation is a treatment for irregular heartbeats, called arrhythmias. It uses heat or cold energy to create tiny scars in the heart. The scars block faulty heart signals and restore a typical heartbeat. Cardiac ablation is most often done using thin, flexible tubes called catheters that are inserted through a blood vessel. Less commonly, ablation is done during heart surgery.

In some embodiments, the database 2313 is configured to include templates and datasets for cardiac device implantation procedures, as illustrated by block 2342. As is recognized by those skilled in the medical fields, cardiac device implantation is the process of inserting a device under the skin to treat the heart. The device is usually placed in the upper left chest for right-handed people, or in the upper right chest for left-handed people.

Data sets in real time for all these procedures may be collected, assembled, labeled, and processed through the neural networks described, to create generative models that can predict outcomes to better assist medical teams ensure favorable outcomes.

Referring now to FIG. 23C, a process is illustrated for assembling multimodal patient-specific data in real time for any one of these specific procedures illustrated above. The process 2343 begins at block 2344, which includes one or more functions or operations for assembling multimodal patient-specific data in real time for any one of these specific cardiovascular procedures. The process 2343 proceeds to the next block 2346, including one or more operations for assembling a subset of multimodal patient-specific data relevant to the cardiovascular procedure. The process 2343 proceeds to the next block 2348, where a particular device required for the procedure is identified and sized. At the same time, an index of vendor devices on the market and their sizes are stored. The process 2343 proceeds to the next block 2350, including one or more functions and operation for executing decisioning algorithms that compare multimodal patient-specific data with the index created. The process 2343 proceeds to the next block 2352, including one or more operations for determining precise measurements of a device appropriate for each specific patient. The process 2343 proceeds to the next block 2354, including one or more operations for anonymizing patient-specific data for storing in a repository. The anonymized data may be compared with specific patient instances to ensure accurate sizing and generate insights for medical team.

Referring now to FIG. 24A, the process for extracting subsets of data in real time is illustrated. The process designated generally by reference numeral 2400, begins at block 2402, which includes one or more functions or operations for extracting data subsets from multimodal patient-specific data in real time from multiple sources. The data subsets may be identified as relevant to a particular procedure. In some embodiments, as illustrated by block 2404, the process identifies data subsets relevant to the TAVR procedures (e.g., aortic valve replacement). The process 2400 proceeds to the next block 2406, including one or more functions and operations for segmenting data sets relevant to select medical parameters identified for each TAVR procedure. The process 2400 proceeds to the next block 2408, including one or more functions for operations for extracting segmented data sets and assembling them in select medical files for processing by defined algorithms and/or medical team/use segmented data sets to train neural networks. The process 2400 proceeds to the next block 2410, including one or more operations for assembling segmented imaging data based on criteria identified in real time to create a composite image/use composite images to train neural networks. The process 2400 proceeds to the next block 2412, including one or more functions or operations for displaying composite images to a medical team during the procedure.

Referring now to FIG. 24B, the process 2413 for assembling data subsets is illustrated. The process 2413 begins at block 2414, including one or more functions or procedures for extracting data subsets from multimodal patient-specific data in real time from multiple sources. The process 2413 proceeds to the next block 2416, including one or more functions or operations for identifying data subsets relevant to cardiovascular procedures. The process 2413 proceeds to the next block 2418, including functions and operation for segmenting data sets relevant to select medical parameters identified for each cardiovascular procedure, to extract segmented data sets and assemble them in select medical files for processing by defined algorithms and/or medical teams. The process 2413 proceeds to the next block 2420, wherein the defined algorithms and medical teams use the segmented data sets to train neural networks. The process 2413 proceeds to the next block 2422, including one or more operations for assembling the segmented imaging data based on criteria identified in real time to create a composite image. The process 2413 proceeds to the next block 2422, including one or more operations for using the composite images to train the neural networks. The process 2413 proceeds to the next block 2424, including one or more operations for displaying the composite image to medical teams during the procedure.

Referring now to FIG. 25A, the process 2500, begins at block 2502, which includes one or more functions or operations for collecting patient-specific data sets relevant to the TAVR procedure in real time. The process 2500 proceeds to the next block 2504, including one or more operations for collecting data sets on devices. In some embodiments, as illustrated by block 2506, this data may be gathered during a procedure that is being performed. The system collects data sets during procedures. The process 2500 proceeds to the next block 2508, including one or more operations for automatically assembling different data sets that have been collected and storing them. The process 2500, proceeds to the next block 2510, including one or more operations for processing and training neural networks to generate compliance reporting data. The process 2500, proceeds to the next block 2512, including one or more operations for assembling and formatting reporting data according to reporting requirements. The process 2500, proceeds to the next block, including one or more operations for assembling and formatting reporting data according to reporting requirements 2512.

Referring now to FIG. 25B, the process 2513 for collecting patient-specific multimodal data in real time is described in more detail. The process 2513 begins at block 2514, including one or more operations for collecting patient-specific data sets in real time relevant to cardiovascular procedures as they are being performed by medical teams. The process 2513 proceeds to the next block 2514 of one or more functions or operations that are configured to collect patient-specific data sets relevant to cardiovascular procedures in real time. The process 2513, proceeds to the next block 2516, including one or more operations for collecting data sets on devices that may be gathered during the procedures. The process 2513, proceeds to the next block 2518, including one or more operations for collecting data sets gathered during the procedure. The process 2513, proceeds to the next block 2520, including one or more operations for automating assembly and storage of different data sets collected. The process 2513, proceeds to the next block 2522, including one or more operations for processing and training neural networks to generate compliance reporting data. The process 2513, proceeds to the next block 2524, including one or more operations for assembling and formatting reporting data according to reporting requirements.

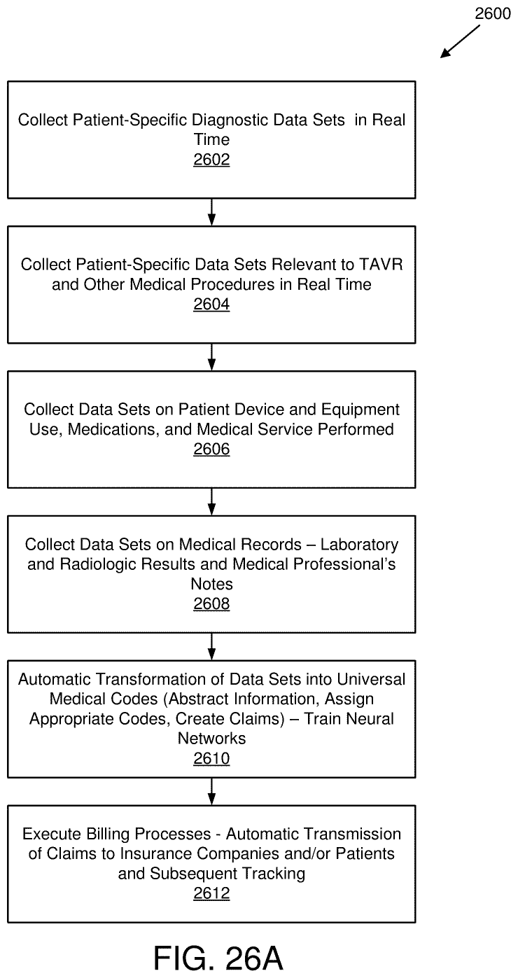

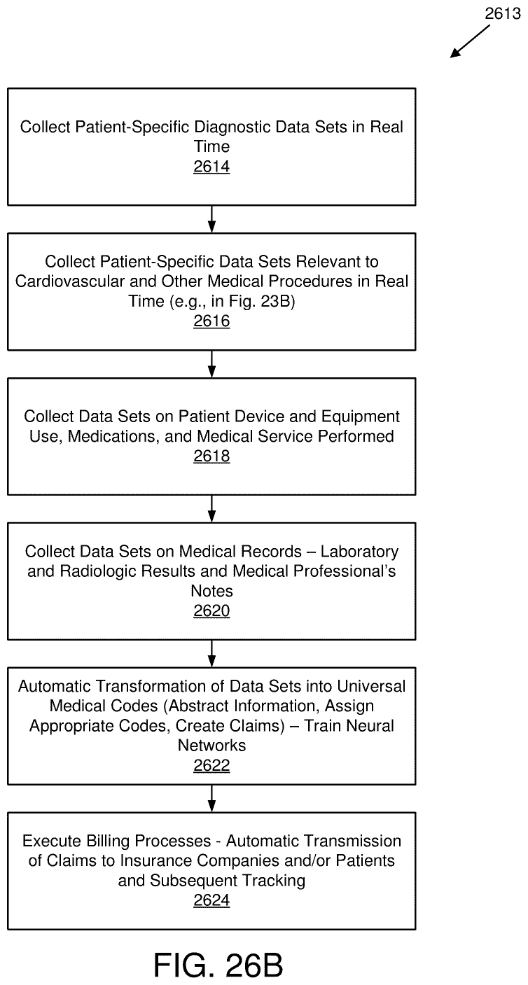

Referring now to FIG. 26A, the process for collecting patient-specific diagnostic data sets is described. In some embodiments, the process illustrated generally at 2600, begins at block 2602, including one or more functions or operations for collecting patient-specific diagnostic data sets in real time. The process 2600 proceeds to the next block 2604, including one or more operations for collecting patient-specific data sets relevant to TAVR and other medical procedures in real time. The process 2600 proceeds to the next block 2606, including one or more operations for collecting data sets on patient device and equipment use, medications, and medical service performed. The process 2600 proceeds to the next block 2608, including one or more operations for collecting data sets on medical records-laboratory and radiologic results and medical professional's notes. The process 2600 proceeds to the next block 2610, including one or more operations for automatically transforming data sets into universal medical codes including generating abstract information, assigning appropriate codes, and creating claims. The neural networks are trained to generate the codes and process the information. The process 2600 proceeds to the next block 2612, including one or more operations for executing billing processes, including automatically transmitting claims to designated insurance companies. In some embodiments, billing may be transmitted to patients. The process 2600 executes all subsequent tracking.

In operation, the billing may include any type of financial information pertaining to costs associated health care services and/or operations of a healthcare facility. For example, the billing data may include costs associated with different procedures and utilization of different resources, including human resources as well as medical instruments and supplies. In some implementations, the billing data may include historical costs, such as historical costs associated with past procedures, courses of care, and the like. For example, with respect to past procedures performed, the billing data may identify total costs (e.g., to the healthcare organization) for each procedure performed, as well as line-item costs associated with individual components of the procedures, including supplies/equipment costs, personnel costs, room/space costs, individual process or procedural steps, and the like. In some embodiments, billing data may also include information regarding reimbursement for respective procedures, including total reimbursement and reimbursement for procedural components. In some implementations, the billing data may also include cost information attributed to loss, procedure complications, and procedural and/or clinical errors (e.g., including cost attributed to litigation associated with the error) etc.

Referring now to FIG. 26B, the processes 2613 are configured to collect patient-specific diagnostic data sets. The processes 2613 begin at block 2614, which includes one or more operations for collecting patient-specific diagnostic data sets in real time. The processes 2613, proceeds to the next block 2616, including one or more operations for collecting patient-specific data sets in real time that are relevant to cardiovascular and other medical procedures. The processes 2613 proceed to the next block 2618, including one or more operations for collecting data sets on patient device and equipment use, medications, and medical service performed. The processes 2613 proceed to the next block 2620, including one or more functions and operations for collecting data sets on medical recording, including laboratory and radiologic results and medical professional's notes. The processes 2613, proceed to the next block 2622, including one or more operations for automatically transforming data sets into universal medical codes. The processes generate abstract information, assign appropriate codes, and create claims. In some embodiments, the processes 2613 train the neural networks.

execute billing processes—automatic transmission of claims to insurance companies and/or patients and subsequent tracking 2624.

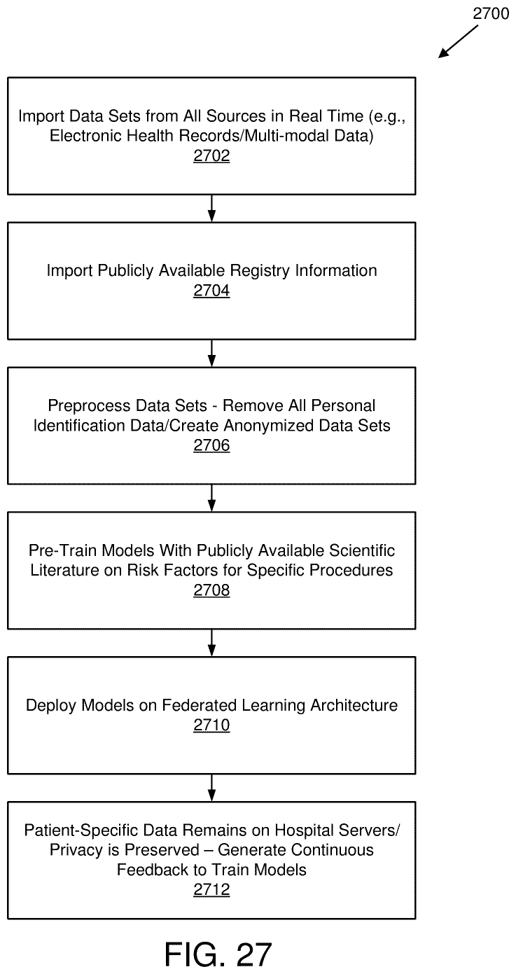

Referring now to FIG. 27, the general process for importing multimodal data and processing the multimodal data is described as illustrated generally by reference numeral 2700. The process 2700 begins at block 2702, including one or more operations for importing data sets from all sources in real time. In some embodiments, the multimodal data includes electronic health records and multimodal data. The process 2700 proceeds to the next block 2704, including one or more functions and operations for importing publicly available registry information. The process 2700 proceeds to the next block 2706, including one or more operations for preprocessing data sets, including function and operations for removing all personal identification data to create anonymized data sets. The process 2700 proceeds to the next block 2708, including one or more operations for using the anonymized data sets to pre-train models with publicly available scientific literature on risk factors for specific procedures. The process 2700 proceeds to the next block 2710, including one or more functions and operations for deploying models on a federated learning architecture. The process 2700 proceeds to the next block 2712, including one or more operations for processing patient-specific data such that the patient-specific data remains on hospital servers and privacy is preserved. The processes and systems generate continuous feedback to train models, as illustrated by block 2712.

The foregoing description of the embodiments of the present implementation has been presented for the purposes of illustration and description. It is not intended to be exhaustive or to limit the present implementation to the precise form disclosed. Many modifications and variations are possible in light of the above teaching. It is intended that the scope of the present inventive technology should not be limited by the detailed description here. As will be understood by those familiar with the art, the present inventive technology may be embodied in other specific forms without departing from the spirit or essential characteristics thereof. Likewise, the particular naming and division of the modules, routines, features, attributes, methodologies and other aspects are not mandatory or significant, and the mechanisms that implement the present inventive technology or its features may have different names, divisions and/or formats. Furthermore, as will be apparent to one of ordinary skills in the relevant art, the modules, routines, features, attributes, methodologies and other aspects of the present inventive technology can be implemented as software, hardware, firmware or any combination of the three. Also, wherever a component, an example of which is a module, of the present inventive technology is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming. Additionally, the present implementation is in no way limited to implementation in any specific programming language, or for any specific operating system or environment. Accordingly, the disclosure of the present inventive technology is intended to be illustrative, but not limiting, of the scope of the present implementation.

What is claimed is:

1. A method for processing multimodal data sets in real time to optimize medical outcomes for a medical procedure, comprising:
compiling a plurality of multimodal patient-specific data in real time obtained from a plurality of multiple sources;
providing a deep learning network model including a plurality of different machine learning layers that are configured to receive the multimodal patient-specific data and additional data including electronic health and medical records, wherein the plurality of machine learning layers are integrated to provide select data subsets for the medical procedure;
training the plurality of different machine learning layers of the deep learning network model with the select data subsets received;
assembling a subset of the multimodal patient-specific data for the medical procedure, wherein the medical procedure is a TAVR procedure;
identifying a trained machine learning model for the TAVR procedure and providing a query for a particular patient medical scenario; and
constructing a predictive outcome summary on the medical procedure for the particular patient medical scenario in real time for a medical professional performing the TAVR procedure.

2. The method of claim 1, wherein the medical procedure is a cardiovascular interventional procedure and wherein the compiling includes compiling an index of a plurality of vendor devices available required for the TAVR procedure and their respective sizes, and executing decisioning algorithms that compare the multimodal patient-specific data with the plurality of vendor devices available, and based on a plurality of precise measurements, selecting a device for a particular patient undergoing the TAVR procedure.

3. The method of claim 1, wherein the additional data further comprises at least one from a group of cardiac and vascular procedures data and electrocardiogram data (ECG) including computed tomography (CT), heart MRI, echocardiogram data, chest x-ray, and angiogram.

4. The method of claim 3, wherein the echocardiogram data includes one from a group of TTE, TEE, and ICE data.

5. The method of claim 1, wherein the patient-specific data is real-time patient image or video data, including one from a group of mobility data, cognitive status data, muscle strength data, speech data, and coordination data.

6. The method of claim 1, wherein the training comprises: receiving raw data sets, preprocessing the raw data sets, creating separate data packets of the raw data sets and labeling the separate data packets, transmitting the raw data packets through a trained network model, assembling the multimodal outputs and displaying the multimodal outputs to the medical professional upon request.

7. The method of claim 1, wherein the training further comprises: generating structured data sets that are delivered to an optimization engine, wherein the optimization engine performs task assessment, task assignment, resource assessment, and applies machine learning algorithms.

8. The method of claim 1, wherein the patient-specific data is encrypted during end-to-end delivery between one or more agents of the deep learning network model.

9. The method of claim 1, wherein the multiple sources of data are data collection portals configured to provide data in real time and synchronously, including payers data, medical imaging data, internal data, billing and coding data, clinician data, video data, publicly available data, electronic medical records, derivative data, regulatory and compliance data, quality and structured reporting data, and social deterministic data.

10. The method of claim 1, wherein structured data sets include imaging data including x-ray data, video data including patient ultrasound and recordings, graphs, tables and text, times series (ECG), sequences (genomics), demographic data, legal and compliance data, and derivative data.

11. The method of claim 1, further comprising:
segmenting the select data subsets based on pattern recognition and correlation of data.

12. A system comprising one or more processors and memory operably coupled with the one or more processors, wherein the memory stores instructions that, in response to execution of the instructions by the one or more processors, cause the one or more processors to perform operations including:
compiling a plurality of multimodal patient-specific data in real time obtained from a plurality of multiple sources;
providing a deep learning network model including a plurality of different machine learning layers that are configured to receive the patient-specific data and additional data including electronic health and medical records, the plurality of different machine learning layers integrated to provide select data subsets for the medical procedure;
training the plurality of different layers of the deep learning network model with the select data subsets;
assembling a subset of the multimodal patient-specific data for the medical procedure, wherein the medical procedure is a TAVR procedure;
identifying a trained machine learning model for the TAVR procedure and providing a query for a particular patient medical scenario; and
constructing a predictive outcome summary on the medical procedure for the patient medical scenario in real time for a medical professional performing the TAVR procedure.

13. The system of claim 12, wherein the medical procedure is a cardiovascular interventional procedure and wherein the compiling includes compiling an index of a plurality of vendor devices available and their respective sizes and executing decisioning algorithms that compare the multimodal patient-specific data with the plurality of vendor devices, and based on a plurality of precise measurements, selecting a device for a particular patient undergoing the TAVR procedure.

14. The system of claim 12, wherein the additional data further comprises at least one from a group of cardiac and vascular procedures data and electrocardiogram data (ECG) including computed tomography (CT), heart MRI, echocardiogram data, chest x-ray, and angiogram.

15. The system of claim 14, wherein the echocardiogram data includes one from a group of TTE, TEE, and ICE data.

16. The system of claim 12, wherein the patient-specific data is real-time patient image or video data, including one from a group of mobility data, cognitive status data, muscle strength data, speech data, and coordination data.

17. The system of claim 12, wherein the training comprises: receiving raw data sets, preprocessing the raw data sets, creating separate data packets of the raw data sets and labeling the separate data packets, transmitting the raw data packets through a trained network model, assembling the multimodal outputs and displaying the multimodal outputs to the medical professional upon request.

18. The system of claim 12, wherein the training further comprises: generating structured data sets that are delivered to an optimization engine, wherein the optimization engine performs task assessment, task assignment, resource assessment, and applies machine learning algorithms.

19. The system of claim 12, wherein the patient-specific data is encrypted during end-to-end delivery between one or more agents of the deep learning network model and wherein the select data subsets are segmented based on pattern recognition and correlation of data.

20. The system of claim 12, wherein the multiple sources of data are data collection portals configured to provide data in real time and synchronously, including payers data, medical imaging data, internal data, billing and coding data, clinician data, video data, publicly available data, electronic medical records, derivative data, regulatory and compliance data, quality and structured reporting data, and social deterministic data and wherein structured data sets include imaging data including x-ray data, video data including patient ultrasound and recordings, graphs, tables and text, times series (ECG), sequences (genomics), demographic data, legal and compliance data, and derivative data.
